(19) United States
(12) Patent Application Publication
(10) Pub. No.: US 2010/0113776 A1
(43) Pub. Date: May 6, 2010

(54) PYRAZOLE DERIVATIVE

(75) Inventors: Toshio Nakamura, Toshima-ku (JP); Dai Nozawa, Toshima-ku (JP); Makoto Tatsuzuki, Toshima-ku (JP); Hiroahi Ohta, Toshima-ku (JP); Takeshi Kuwada, Toshima-ku (JP); Tomoko Ishizaka, Toshima-ku (JP); Tomoko Tamita, Toshima-ku (JP); Seiji Masuda, Toshima-ku (JP)

Correspondence Address: SUGHRUE MION, PLLC, 2100 PENNSYLVANIA AVENUE, N.W., SUITE 800, WASHINGTON, DC, 20037, US

(73) Assignee: TAISHO PHARMACEUTICAL CO.,LTD., Toshima-ku, Tokyo (JP)

(21) Appl. No.: 12/519,083

(22) PCT Filed: Dec. 14, 2007
(86) PCT No.: PCT/JP2007/074092

§ 371 Date: Jun. 12, 2009

(30) Foreign Application Priority Data
Dec. 14, 2006 (JP) 2006-336506
Jul. 6, 2007 (JP) 2007-178839

(51) Int. Cl. C07D 401/14; C07D 401/12; C07D 413/14; C07D 403/14

(52) U.S. Cl. 544/130; 546/211; 546/187; 544/364; 548/364.1

(57) ABSTRACT

A novel pyrazole derivative of the following formula having a histamine H3 receptor antagonistic effect:

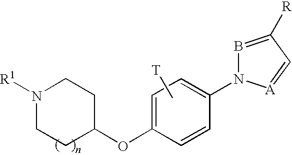

or a pharmaceutically acceptable salt thereof or a pharmaceutical preparation comprising the same as an active ingredient is effective for prevention or treatment of dementia, Alzheimer's disease, attention-deficit hyperactivity disorder, schizophrenia, eating disorders, obesity, diabetes, hyperlipidemia, sleep disorders, narcolepsy, sleep apnea syndrome, circadian rhythm disorder, depression, allergic rhinitis or other diseases.

### TECHNICAL FIELD

The present invention relates to pyrazole derivatives having a histamine H3 receptor antagonistic effect, as well as pharmaceutical preparations comprising such a derivative as an active ingredient, more particularly prophylactic or therapeutic agents for dementia, Alzheimer's disease, attention-deficit hyperactivity disorder, schizophrenia, epilepsy, central convulsion, eating disorders, obesity, diabetes, hyperlipidemia, sleep disorders, narcolepsy, sleep apnea syndrome, circadian rhythm disorder, depression or allergic rhinitis.

### BACKGROUND ART

Histamine is usually stored within intracellular granules in mast cells, lung, liver and gastric mucosa, etc. In response to external stimuli such as antigen binding to cell surface antibody, histamine is released into the extracellular environment. For example, when mast cells are stimulated by an antigen entering from outside, histamine is released from the mast cells and stimulates histamine H1 (H1) receptors located on blood vessels or smooth muscle to cause allergic reactions. Likewise, histamine released from ECL cells (enterochromaffin-like cells) on the gastric mucosa stimulates histamine H2 (H2) receptors on the parietal cells to promote gastric acid secretion. Based on these facts, H1 and H2 receptor antagonists have been developed as therapeutic agents for allergic diseases and gastric ulcer, respectively, both of which are now used widely as medicaments.

Further, it has been elucidated that histamine serves as a neurotransmitter and acts on the third histamine receptor (histamine H3 (H3) receptor) located in central and peripheral nerves to thereby exert various physiological functions. This receptor was cloned in 1999 and determined for its gene sequence and amino acid sequence. However, its amino acid sequence homology was as low as 22% and 21.4% with H1 receptor and H2 receptor, respectively (see Non-patent Document 1). H3 receptors are present in the presynaptic membrane and are shown to serve as autoreceptors controlling the synthesis and release of histamine (see Non-patent Document 2). Moreover, H3 receptors are also shown to control not only the release of histamine, but also the release of other neurotransmitters including acetylcholine, serotonin, dopamine and noradrenaline (see Non-patent Document 3). These facts suggest that H3 receptor antagonists may serve as therapeutic agents for various diseases related to abnormal release of neurotransmitters in the nerves.

The results of animal model studies using synthetic compounds indicate a possibility that among H3 receptor antagonists, H3 receptor antagonists can be used as therapeutic agents for dementia, Alzheimer's disease (see Non-patent Documents 4 and 5), attention-deficit hyperactivity disorder (see Non-patent Document 6), schizophrenia (see Non-patent Document 7), epilepsy, central convulsion, etc.

Moreover, it is shown that H3 receptors are involved in feeding behavior (see Non-patent Document 8); and hence possible target diseases for H3 receptor antagonists also include metabolic diseases such as eating disorders, obesity, diabetes, hyperlipidemia, etc.

Further, it is shown that histamine regulates the circadian rhythm in the brain and is responsible for maintaining a balance between waking and sleeping states (see Non-patent Documents 9 and 10); and hence possible target diseases for H3 receptor antagonists also include sleep disorders and diseases associated with sleep disorders such as narcolepsy, sleep apnea syndrome, circadian rhythm disorder, depression, etc.

Furthermore, it is shown that H3 receptors are present in sympathetic nerves on the nasal mucosa, and there is a report showing that the combined use of H3 and H1 receptor antagonists remarkably improves nasal congestion (see Non-patent Document 11). This indicates a possibility that H3 receptor antagonists are useful for treatment of allergic rhinitis or other diseases, either alone or in combination with H1 receptor antagonists.

H3 receptor antagonists have been summarized in several reviews (see Non-patent Documents 12 to 15), and reference may be made to these reviews. In the early years, many reports were issued for imidazole compounds starting from histamine itself as a leading compound. However, these compounds have not yet been developed as medicaments because they are feared to have negative effects such as inhibition of a drug-metabolizing enzyme, cytochrome P450 (CYP).

In recent years, many documents and patents have been reported for non-imidazole H3 receptor antagonists (see Patent Documents 1 to 10).

For example, Patent Document 11 reports a histamine H3 receptor antagonist having a structure represented by the following formula (A), in which a benzene ring is attached to a pyrrole ring.

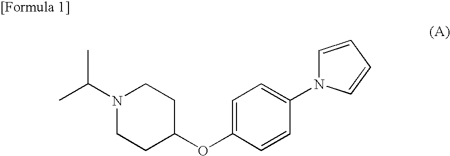

Patent Document 12 reports a histamine H3 receptor antagonist having a biphenyl structure represented by the following formula (B).

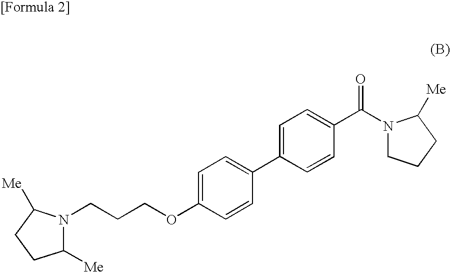

Patent Document 13 reports a histamine H3 receptor antagonist having a structure represented by the following formula (C), in which a pyrimidine ring is attached to a benzene ring.

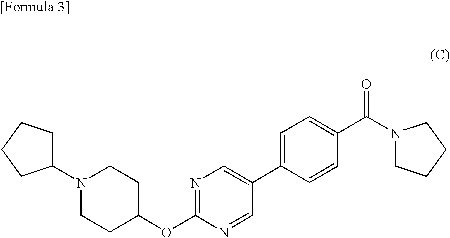

Patent Document 14 reports a histamine H3 receptor antagonist having a structure represented by the following formula (D), in which a benzene ring is attached to a pyrazole ring.

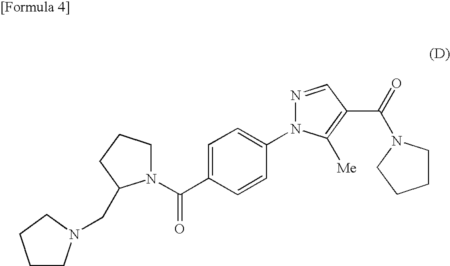

However, there is no report about compounds having the structure disclosed in the present invention.

Patent Document 1: International Patent Publication No. WO2005/097751

Patent Document 2: International Patent Publication No. WO2005/097778

Patent Document 3: International Patent Publication No. WO2005/118547

Patent Document 4: International Patent Publication No. WO2006/014136

Patent Document 5: International Patent Publication No. WO2006/045416

Patent Document 6: International Patent Publication No. WO2006/046131

Patent Document 7: International Patent Publication No. WO2006/059778

Patent Document 8: International Patent Publication No. WO2006/061193

Patent Document 9: International Patent Publication No. WO2006/107661

Patent Document 10: International Patent Publication No. WO2006/103057

Patent Document 11: International Patent Publication No. WO2002/012190

Patent Document 12: International Patent Publication No. WO2002/040461

Patent Document 13: International Patent Publication No. WO2005/007644

Patent Document 14: International Patent Publication No. WO2006/023462

Non-patent Document 1: Lovenberg T. W. et al., Molecular pharmacology, 55, 1101-1107, 1999

Non-patent Document 2: Arrang J-M. et al., Nature, 302, 832-837, 1983

Non-patent Document 3: Brown R. E. et al., Progress in Neurobiology, 63, 637-672, 2001

Non-patent Document 4: Huang Y-W. et al., Behavioural Brain Research, 151, 287-293, 2004

Non-patent Document 5: Komater V. A. et al., Behavioural Brain Research, 159, 295-300, 2005

Non-patent Document 6: Passani M. B. et al., Neuroscience and Biobehavioral Reviews, 24, 107-113, 2000

Non-patent Document 7: Fox G. B. et al., J. Pharmacol. Exp. Ther., 313, 176-190, 2005

Non-patent Document 8: Hancock A. A. et al., Curr. Opin. Investig. Drug, 4, 1190-1197

Non-patent Document 9: Huang Z-L. et al., Prog. Natr. Acad. Sci., 103, 4687-4692, 2006

Non-patent Document 10: Babier A. J. et al., Br. J. Pharmacol., 143, 649-661, 2004

Non-patent Document 11: McLeod R. L. et al., Am. J. Rhinol., 13, 391-399, 1999

Non-patent Document 12: Schwartz J. C. et al., Trends in Pharmacol. Sci., 7, 24-28, 1986

Non-patent Document 13: Passani M. B. et al., Trends in Pharmacol. Sci., 25, 618-625, 2004

Non-patent Document 14: Leurs R. et al., Nature Drug Discovery, 4, 107-122, 2005

Non-patent Document 15: Leurs R. et al., Drug Discovery Today, 10, 1613-1627, 2005

### DISCLOSURE OF THE INVENTION

### Problems to be Solved by the Invention

The object of the present invention is to provide novel pyrazole derivatives, more specifically novel pyrazole derivatives which have a histamine H3 receptor antagonistic effect and are useful for prevention and treatment of histamine H3 receptor-mediated disorders such as dementia, Alzheimer's disease, attention-deficit hyperactivity disorder, schizophrenia, epilepsy, central convulsion, eating disorders, obesity, diabetes, hyperlipidemia, sleep disorders, narcolepsy, sleep apnea syndrome, circadian rhythm disorder, depression, allergic rhinitis or other diseases.

### Means for Solving the Problems

As a result of extensive and intensive efforts made to achieve the above object, the inventors of the present invention have found that pyrazole derivatives have a strong antagonistic effect on histamine H3 receptors. This finding led to the completion of the present invention.

Namely, the present invention is directed to the following.

1) A pyrazole derivative represented by formula (I) or a pharmaceutically acceptable salt thereof:

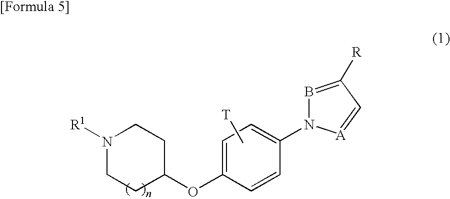

{wherein

A and B, which are different from each other, each represent a carbon atom or a nitrogen atom,

R¹ represents C₁-C₆ alkyl (wherein said C₁-C₆ alkyl may be substituted with C₃-C₈ cycloalkyl or hydroxyl); C₃-C₈ cycloalkyl (wherein said C₃-C₈ cycloalkyl may be substituted with C₁-C₆ alkyl or hydroxyl) or C₃-C₈ alkenyl,

n represents an integer of 0 to 2,

T represents a hydrogen atom; halogen or C₁-C₆ alkyl,

R represents a group represented by any of the following structural formulae (I) to (VI):

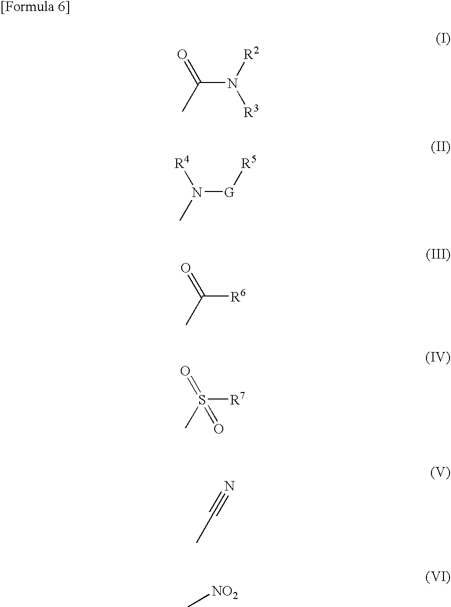

R² and R³, which may be the same or different, each represent a hydrogen atom; C₁-C₆ alkyl (wherein said C₁-C₆ alkyl may be substituted with halogen; C₃-C₈ cycloalkyl; hydroxyl; C₂-C₇ alkoxycarbonyl or carboxy); C₃-C₈ cycloalkyl (wherein said C₃-C₈ cycloalkyl may be substituted with halogen; C₁-C₆ alkyl or hydroxyl) or a group represented by —(CH₂)ₘ—Ar, or

R² and R³ are attached to each other together with their adjacent nitrogen atom to form a 4- to 7-membered saturated heterocyclic ring which may contain, as its ring members, one or more nitrogen, oxygen or sulfur atoms in addition to said adjacent nitrogen atom (wherein said saturated heterocyclic ring may be substituted with halogen; C₁-C₆ alkyl; C₁-C₆ alkoxy or hydroxyl),

Ar represents aryl (wherein said aryl may be substituted with halogen; C₁-C₆ alkyl; C₁-C₆ alkoxy; hydroxyl; C₂-C₇ alkoxycarbonyl; cyano; C₂-C₇ alkylaminocarbonyl; C₃-C₁₃ dialkylaminocarbonyl; carbamoyl or carbonyl which is attached via a ring nitrogen atom to a 3- to 7-membered saturated heterocyclic ring which has at least one nitrogen atom as its ring member and may contain one or more additional nitrogen, oxygen or sulfur atoms) or heteroaryl (wherein said heteroaryl may be substituted with halogen; C₁-C₆ alkyl; C₁-C₆ alkoxy; hydroxyl; C₂-C₇ alkoxycarbonyl; cyano; C₂-C₇ alkylaminocarbonyl; C₃-C₁₃ dialkylaminocarbonyl; carbamoyl or carbonyl which is attached via a ring nitrogen atom to a 3- to 7-membered saturated heterocyclic ring which has at least one nitrogen atom as its ring member and may contain one or more additional nitrogen, oxygen or sulfur atoms),

m represents an integer of 0 to 2,

G represents —CO— or —SO₂—,

R⁴ represents a hydrogen atom or C₁-C₆ alkyl,

R⁵ represents C₁-C₆ alkyl (wherein said C₁-C₆ alkyl may be substituted with halogen; C₃-C₈ cycloalkyl; C₁-C₆ alkoxy or hydroxyl); C₃-C₈ cycloalkyl (wherein said C₃-C₈ cycloalkyl may be substituted with halogen; C₁-C₆ alkyl; C₁-C₆ alkoxy or hydroxyl); aryl (wherein said aryl may be substituted with halogen; alkyl; C₁-C₆ alkoxy; hydroxyl or cyano) or heteroaryl (wherein said heteroaryl may be substituted with halogen; alkyl; C₁-C₆ alkoxy; hydroxyl or cyano), or

R⁴ and R⁵ may be attached to each other together with their adjacent nitrogen atom and carbonyl carbon to form a 5- to 7-membered saturated heterocyclic ring which may contain, as its ring members, one or more nitrogen, oxygen or sulfur atoms in addition to said adjacent nitrogen atom (wherein said saturated heterocyclic ring may be substituted with halogen; C₁-C₆ alkyl; C₁-C₆ alkoxy; hydroxyl or oxo),

R⁶ represents C₁-C₆ alkyl; C₃-C₈ cycloalkyl; C₁-C₆ alkoxy; aryl (wherein said aryl may be substituted with halogen; alkyl; C₁-C₆ alkoxy; hydroxyl or cyano) or heteroaryl (wherein said heteroaryl may be substituted with halogen; C₁-C₆ alkyl; C₁-C₆ alkoxy; hydroxyl or cyano), and

R⁷ represents C₁-C₆ alkyl; C₁-C₆ alkoxy; amino; C₁-C₆ alkylamino; C₂-C₁₂ dialkylamino; a 4- to 7-membered saturated heterocyclic ring (wherein said saturated heterocyclic ring may be substituted with halogen; alkyl; C₁-C₆ alkoxy; hydroxyl or cyano); aryl (wherein said aryl may be substituted with halogen; alkyl; C₁-C₆ alkoxy; hydroxyl or cyano) or heteroaryl (wherein said heteroaryl may be substituted with halogen; C₁-C₆ alkyl; C₁-C₆ alkoxy; hydroxyl or cyano)}.

2) The pyrazole derivative or pharmaceutically acceptable salt thereof according to 1) above, which is represented by formula (I) wherein R is represented by structural formula (I).
3) A compound selected from the group consisting of:
1-cyclopentyl-4-{4-[4-(pyrrolidin-1-ylcarbonyl)-1H-pyrazol-1-yl]phenoxy}piperidine,
1-ethyl-4-{4-[4-(pyrrolidin-1-ylcarbonyl)-1H-pyrazol-1-yl]phenoxy}piperidine,
1-(1-methylethyl)-4-{4-[4-(pyrrolidin-1-ylcarbonyl)-1H-pyrazol-1-yl]phenoxy}piperidine,
1-cyclohexyl-4-{4-[4-(pyrrolidin-1-ylcarbonyl)-1H-pyrazol-1-yl]phenoxy}piperidine,
1-(2-methylcyclopentyl)-4-{4-[4-(pyrrolidin-1-ylcarbonyl)-1H-pyrazol-1-yl]phenoxy}piperidine,
1-(cyclopropylmethyl)-4-{4-[4-(pyrrolidin-1-ylcarbonyl)-1H-pyrazol-1-yl]phenoxy}piperidine,
1-(3-methyl-2-buten-1-yl)-4-{4-[4-(pyrrolidin-1-ylcarbonyl)-1H-pyrazol-1-yl]phenoxy}piperidine,
1-(cyclobutylmethyl)-4-{4-[4-(pyrrolidin-1-ylcarbonyl)-1H-pyrazol-1-yl]phenoxy}piperidine,
1-(cyclopentylmethyl)-4-{4-[4-(pyrrolidin-1-ylcarbonyl)-1H-pyrazol-1-yl]phenoxy}piperidine,
2-(4-{4-[4-(pyrrolidin-1-ylcarbonyl)-1H-pyrazol-1-yl]phenoxy}piperidin-1-yl)ethanol,
3-(4-{-4-[4-(pyrrolidin-1-ylcarbonyl)-1H-pyrazol-1-yl]phenoxy}piperidin-1-yl)cyclopentanol,
ethyl 1-{4-[(1-cyclobutylpiperidin-4-yl)oxy]phenyl}-1H-pyrazole-4-carboxylic acid,
ethyl 1-{4-[(1-cyclopentylpiperidin-4-yl)oxy]phenyl}-1H-pyrazole-4-carboxylic acid,
1-cyclobutyl-4-{4-[4-(pyrrolidin-1-ylcarbonyl)-1H-pyrazol-1-yl]phenoxy}piperidine,
1-[(1-{4-[(1-cyclopentylpiperidin-4-yl)oxy]phenyl}-1H-pyrazol-4-yl)carbonyl]pyrrolidin-3-ol,
1-{4-[(1-cyclobutylpiperidin-4-yl)oxy]phenyl}-N-cyclopentyl-1H-pyrazole-4-carboxamide,
1-{4-[(1-cyclobutylpiperidin-4-yl)oxy]phenyl}-N-methyl-1H-pyrazole-4-carboxamide,
1-{4-[(1-cyclobutylpiperidin-4-yl)oxy]phenyl}-N-(2,2,2-trifluoroethyl)-1H-pyrazole-4-carboxamide,
1-{4-[(1-cyclobutylpiperidin-4-yl)oxy]phenyl}-N-(cyclohexylmethyl)-1H-pyrazole-4-carboxamide,
1-cyclobutyl-4-{4-[4-(piperidin-1-ylcarbonyl)-1H-pyrazol-1-yl]phenoxy}piperidine,
1-cyclobutyl-4-(4-{4-[(2-methylpyrrolidin-1-yl)carbonyl-1H-pyrazol-1-yl}phenoxy)piperidine,
1-{4-[(1-cyclobutylpiperidin-4-yl)oxy]phenyl}-N,N-diethyl-1H-pyrazole-4-carboxamide,
N-tert-butyl-1-{4-[(1-cyclobutylpiperidin-4-yl)oxy]phenyl}-1H-pyrazole-4-carboxamide,
1-{4-[(1-cyclobutylpiperidin-4-yl)oxy]phenyl}-N,N-dimethyl-1H-pyrazole-4-carboxamide,
4-[(1-{4-[(1-cyclobutylpiperidin-4-yl)oxy]phenyl}-1H-pyrazol-4-yl)carbonyl]morpholine,
1-[(1-{4-[(1-cyclobutylpiperidin-4-yl)oxy]phenyl}-1H-pyrazol-4-yl)carbonyl]-4-methylpiperazine,
1-{4-[(1-cyclobutylpiperidin-4-yl)oxy]phenyl}-N-cyclohexyl-N-methyl-1H-pyrazole-4-carboxamide,
1-{-4-[(1-cyclobutylpiperidin-4-yl)oxy]phenyl}-N-methyl-N-(2-methylpropyl)-1H-pyrazole-4-carboxamide,
1-cyclobutyl-4-(4-{4-[(3,3-difluoropyrrolidin-1-yl)carbonyl]-1H-pyrazol-1-yl}phenoxy)piperidine,
1-{4-[(1-cyclobutylpiperidin-4-yl)oxy]phenyl}-N-(4-fluorophenyl)-1H-pyrazole-4-carboxamide,
1-{4-[(1-cyclobutylpiperidin-4-yl)oxy]phenyl}-N-(4-fluorobenzyl)-1H-pyrazole-4-carboxamide,
4-{4-[4-(azetidin-1-ylcarbonyl)-1H-pyrazol-1-yl]phenoxy}-1-cyclopentylpiperidine,
4-[(1-{4-[(1-cyclopentylpiperidin-4-yl)oxy]phenyl}-1H-pyrazol-4-yl)carbonyl]morpholine,
1-cyclopentyl-4-(4-{4-[(3,3-difluoropyrrolidin-1-yl)carbonyl]-1H-pyrazol-1-yl}phenoxy)piperidine,
N-cyclopentyl-1-{-4-[(1-cyclopentylpiperidin-4-yl)oxy]phenyl}-1H-pyrazole-4-carboxamide,
N-cyclohexyl-1-{4-[(1-cyclopentylpiperidin-4-yl)oxy]phenyl}-1H-pyrazole-4-carboxamide,
N-benzyl-1-{4-[(1-cyclopentylpiperidin-4-yl)oxy]phenyl}-1H-pyrazole-4-carboxamide,
1-{4-[(1-cyclopentylpiperidin-4-yl)oxy]phenyl}-N-(4-fluorobenzyl)-1H-pyrazole-4-carboxamide,
1-{4-[(1-cyclopentylpiperidin-4-yl)oxy]phenyl}-N-(3-fluorobenzyl)-1H-pyrazole-4-carboxamide,
1-{4-[(1-cyclopentylpiperidin-4-yl)oxy]phenyl}-N-(2-fluorobenzyl)-1H-pyrazole-4-carboxamide,
1-{4-[(1-cyclopentylpiperidin-4-yl)oxy]phenyl}-N-phenyl-1H-pyrazole-4-carboxamide,
1-{4-[(1-cyclopentylpiperidin-4-yl)oxy]phenyl}-N-(4-fluorophenyl)-1H-pyrazole-4-carboxamide,
1-{4-[(1-cyclopentylpiperidin-4-yl)oxy]phenyl}-N-(4-methylphenyl)-1H-pyrazole-4-carboxamide,
1-{4-[(1-cyclopentylpiperidin-4-yl)oxy]phenyl}-N-(4-methoxyphenyl)-1H-pyrazole-4-carboxamide,
1-{4-[(1-cyclopentylpiperidin-4-yl)oxy]phenyl}-N-[4-(pyrrolidin-1-ylcarbonyl)phenyl]-1H-pyrazole-4-carboxamide,
N-cyclopentyl-1-{4-[(1-cyclopentylpiperidin-4-yl)oxy]phenyl}-N-methyl-1H-pyrazole-4-carboxamide,
ethyl 4-{[(1-{4-[(1-cyclopentylpiperidin-4-yl)oxy]phenyl}-1H-pyrazol-4-yl)carbonyl]amino}butanoic acid,
tert-butyl 4-{[(1-{4-[(1-cyclopentylpiperidin-4-yl)oxy]phenyl}-1H-pyrazol-4-yl]carbonyl}amino]butanoic acid,
4-{[(1-{4-[(1-cyclopentylpiperidin-4-yl)oxy]phenyl}-1H-pyrazol-4-yl)carbonyl]amino]butanoic acid,
1-tert-butyl-4-{4-[4-(pyrrolidin-1-ylcarbonyl)-1H-pyrazol-1-yl]phenoxy}piperidine,
1-{4-[(1-cyclopropylpiperidin-4-yl)oxy]phenyl}-N-(4-fluorophenyl)-1H-pyrazole-4-carboxamide,
N-(4-fluorophenyl)-1-(4-{[1-(1-methylethyl)piperidin-4-yl]oxy}phenyl)-1H-pyrazole-4-carboxamide,
1-{4-[(1-cyclopentylpyrrolidin-3-yl)oxy]phenyl}-4-(pyrrolidin-1-ylcarbonyl)-1H-pyrazole,
1-{4-[(1-cyclobutylpyrrolidin-3-yl)oxy]phenyl}-4-(pyrrolidin-1-ylcarbonyl)-1H-pyrazole,
1-cyclopentyl-4-{3-fluoro-4-[4-(pyrrolidin-1-ylcarbonyl)-1H-pyrazol-1-yl]phenoxy}piperidine,
1-cyclobutyl-4-{3-fluoro-4-[4-(pyrrolidin-1-ylcarbonyl)-1H-pyrazol-1-yl]phenoxy}piperidine,
1-cyclopentyl-4-{2-fluoro-4-[4-(pyrrolidin-1-ylcarbonyl)-1H-pyrazol-1-yl]phenoxy}piperidine,
1-cyclobutyl-4-{2-fluoro-4-[4-(pyrrolidin-1-ylcarbonyl)-1H-pyrazol-1-yl]phenoxy}piperidine,
1-cyclopentyl-4-{3-methyl-4-[4-(pyrrolidin-1-ylcarbonyl)-1H-pyrazol-1-yl]phenoxy}piperidine,
1-cyclobutyl-4-{3-methyl-4-[4-(pyrrolidin-1-ylcarbonyl)-1H-pyrazol-1-yl]phenoxy}piperidine,
1-{4-[(1-cyclobutylpiperidin-4-yl)oxy]phenyl}-1H-pyrazole-4-carboxamide,
1-{4-[(1-cyclopentylpiperidin-4-yl)oxy]phenyl}-1H-pyrazole-4-carbonitrile,
1-cyclobutyl-4-[4-(4-nitro-1H-pyrazol-1-yl)phenoxy]piperidine,
4-chloro-N-(1-{4-[(1-cyclobutylpiperidin-4-yl)oxy]phenyl}-1H-pyrazol-4-yl)butaneamide,
1-(1-{4-[(1-cyclopentylpiperidin-4-yl)oxy]phenyl}-1H-pyrazol-4-yl)pyrrolidin-2-one,
2-chloroethyl {1-[4-(1-cyclobutylpiperidin-4-yloxy)phenyl]-1H-pyrazol-4-yl}-carbamic acid,
3-(1-{4-[(1-cyclopentylpiperidin-4-yl)oxy]phenyl}-1H-pyrazol-4-yl)-1,3-oxazolidin-2-one, and
{1-[4-(1-cyclobutylpiperidin-4-yloxy)phenyl]-1H-pyrazol-3-yl}pyrrolidin-1-yl-methanone.
   4) A pharmaceutical preparation, which comprises the pyrazole derivative or pharmaceutically acceptable salt thereof according to any one of 1) to 3) above as an active ingredient.
   5) The pharmaceutical preparation according to 4) above, which is a histamine H3 receptor antagonist.
   6) A prophylactic or therapeutic agent for dementia, Alzheimer's disease, attention-deficit hyperactivity disorder, schizophrenia, epilepsy, central convulsion, eating disorders, obesity, diabetes, hyperlipidemia, sleep disorders, narcolepsy, sleep apnea syndrome, circadian rhythm disorder, depression or allergic rhinitis, which comprises the pyrazole derivative or pharmaceutically acceptable salt thereof according to any one of 1) to 3) above as an active ingredient.

### ADVANTAGES OF THE INVENTION

The compounds of the present invention were found to have an excellent histamine H3 receptor antagonistic effect.

### BEST MODE FOR CARRYING OUT THE INVENTION

The terms and expressions used herein are defined as follows.

As used herein, the term “halogen” refers to a fluorine atom, a chlorine atom, a bromine atom or an iodine atom.

The term “C₁-C₆ alkyl” refers to a linear or branched alkyl group containing 1 to 6 carbon atoms. Examples include methyl, ethyl, n-propyl, isopropyl, n-butyl, isobutyl, sec-butyl, tert-butyl, n-pentyl, isopentyl, neopentyl and n-hexyl groups.

The term “C₃-C₈ cycloalkyl” refers to a cyclopropyl, cyclobutyl, cyclopentyl, cyclohexyl, cycloheptyl or cyclooctyl group.

The term “C₃-C₈ alkenyl” refers to a linear or branched alkenyl group containing 3 to 8 carbon atoms. Examples include 1-propen-2-yl, 1-buten-3-yl, 1-hexen-5-yl, 1-octen-7-yl, 1-(3-methylbuten-2-yl), 1-hexen-2-yl and 1-(3,4-dimethylpenten-3-yl) groups.

The term “C₁-C₆ alkoxy” refers to a linear or branched alkoxy group containing 1 to 6 carbon atoms. Examples include methoxy, ethoxy, n-propoxy, isopropoxy, n-butoxy, isobutoxy, sec-butoxy, tert-butoxy, n-pentyloxy, isopentyloxy, neopentyloxy and n-hexyloxy groups.

The term “C₁-C₆ alkylamino” refers to an amino group substituted with a linear or branched alkyl group containing 1 to 6 carbon atoms. Examples include methylamino, ethylamino, n-propylamino, isopropylamino, n-butylamino, isobutylamino, sec-butylamino, tert-butylamino, n-pentylamino, isopentylamino, neopentylamino and n-hexylamino groups.

The term “C₂-C₁₂ dialkylamino” refers to an amino group substituted with two linear or branched alkyl groups each containing 1 to 6 carbon atoms. Examples include dimethylamino, diethylamino, di-n-propylamino, N,N-isopropylmethylamino, di-n-butylamino, diisobutylamino, N,N-sec-butylethylamino, N,N-tert-butylmethylamino, di-n-pentylamino, N,N-isopentylmethylamino, N,N-neopentylmethylamino and di-n-hexylamino groups.

The term “4- to 7-membered saturated heterocyclic ring” refers to a saturated 4- to 7-membered cyclic amino group. Examples include 1-azetidyl, 1-pyrrolidyl, piperidino and 1-azepanyl groups.

The term “C₂-C₇ alkoxycarbonyl” refers to a carbonyl group attached to a linear or branched alkoxy group containing 1 to 6 carbon atoms. Examples include methoxycarbonyl, ethoxycarbonyl, n-propoxycarbonyl, isopropoxycarbonyl, n-butoxycarbonyl, isobutoxycarbonyl, sec-butoxycarbonyl, tert-butoxycarbonyl, n-pentyloxycarbonyl, isopentyloxycarbonyl, neopentyloxycarbonyl and n-hexyloxycarbonyl groups.

The term “C₂-C₇ alkylaminocarbonyl” refers to a carbonyl group attached to an amino group substituted with a linear or branched alkyl group containing 1 to 6 carbon atoms. Examples include methylaminocarbonyl, ethylaminocarbonyl, n-propylaminocarbonyl, isopropylaminocarbonyl, n-butylaminocarbonyl, isobutylaminocarbonyl, sec-butylaminocarbonyl, tert-butylaminocarbonyl, n-pentylaminocarbonyl, isopentylaminocarbonyl, neopentylaminocarbonyl and n-hexylaminocarbonyl groups.

The term “C₃-C₁₃ dialkylaminocarbonyl” refers to a carbonyl group attached to an amino group substituted with two linear or branched alkyl groups each containing 1 to 6 carbon atoms. Examples include dimethylaminocarbonyl, diethylaminocarbonyl, di-n-propylaminocarbonyl, N,N-isopropylmethylaminocarbonyl, di-n-butylaminocarbonyl, diisobutylaminocarbonyl, N,N-sec-butylethylaminocarbonyl, N,N-tert-butylmethylaminocarbonyl, di-n-pentylaminocarbonyl, N,N-isopentylmethylaminocarbonyl, N,N-neopentylmethylaminocarbonyl and di-n-hexylaminocarbonyl groups.

The expression “carbonyl which is attached via a ring nitrogen atom to a 3- to 7-membered saturated heterocyclic ring which has at least one nitrogen atom as its ring member and may contain one or more additional nitrogen, oxygen or sulfur atoms” is intended to mean a carbonyl group which is attached via a ring nitrogen atom to a saturated 3- to 7-membered monocyclic heterocyclic ring which contains one nitrogen atom and may further contain one or more additional heteroatoms selected from nitrogen, oxygen and sulfur atoms. Examples include aziridine-1-carbonyl, azetidine-1-carbonyl, pyrrolidine-1-carbonyl, piperidine-1-carbonyl, azepane-1-carbonyl, imidazolidine-1-carbonyl, pyrazolidine-1-carbonyl, piperazine-1-carbonyl, oxazolidine-1-carbonyl, morpholine-1-carbonyl and thiomorpholine-1-carbonyl groups.

The expression “attached to each other together with their adjacent nitrogen atom to form a 4- to 7-membered saturated heterocyclic ring which may contain, as its ring members, one or more nitrogen, oxygen or sulfur atoms in addition to said adjacent nitrogen atom” is intended to mean a saturated 4- to 7-membered monocyclic heterocyclic group which contains one nitrogen atom and may further contain one or more additional heteroatoms selected from nitrogen, oxygen and sulfur atoms. Examples include 1-azetidyl, 1-pyrrolidinyl, piperidino, 1-azepanyl, 1-imidazolidinyl, 1-pyrazolidinyl, 1-piperazinyl, 3-oxazolidinyl, morpholino and 1-thiomorpholinyl groups.

The term “aryl” refers to a phenyl group or a naphthyl group.

The term “heteroaryl” refers to a monocyclic or bicyclic aromatic heterocyclic group. Examples include pyridine, pyridazine, pyrimidine, pyrazine, quinoline, isoquinoline, quinazoline, quinoxaline, pyrrole, furan, thiophene, pyrazole, imidazole, oxazole, isoxazole, thiazole, isothiazole, triazole, indole, benzofuran, benzothiophene, benzoimidazole, indazole, benzoxazole, benzothiazole and benzotriazole groups.

As used herein, the term “pharmaceutically acceptable salt” is intended to include a salt with an inorganic acid such as sulfuric acid, hydrochloric acid, hydrobromic acid, phosphoric acid or nitric acid; a salt with an organic acid such as acetic acid, oxalic acid, lactic acid, tartaric acid, fumaric acid, maleic acid, citric acid, benzenesulfonic acid, methanesulfonic acid, p-toluenesulfonic acid, benzoic acid, camphorsulfonic acid, ethanesulfonic acid, glucoheptonic acid, gluconic acid, glutamic acid, glycolic acid, malic acid, malonic acid, mandelic acid, galactaric acid or naphthalene-2-sulfonic acid; a salt with one or more metal ions such as lithium ion, sodium ion, potassium ion, calcium ion, magnesium ion, zinc ion and/or aluminum ion; as well as a salt with ammonia or an amine such as arginine, lysin, piperazine, choline, diethylamine, 4-phenylcyclohexylamine, 2-aminoethanol or benzathine.

The compounds of the present invention may be present in the form of various solvates. They may also be in hydrate form in terms of applicability as pharmaceutical preparations.

The compounds of the present invention encompass all of the following: enantiomers, diastereomers, equilibrium compounds, mixtures thereof at any ratio, racemates, etc.

The compounds of the present invention also encompass compounds in which one or more hydrogen atoms, carbon atoms, nitrogen atoms, oxygen atoms or sulfur atoms are replaced with their radioisotopes or stable isotopes. These labeled compounds are useful for metabolism and/or pharmacokinetics study, biological analysis as receptor ligands or other purposes.

The compounds of the present invention may be formulated into pharmaceutical preparations in combination with one or more pharmaceutically acceptable carriers, excipients or diluents. Examples of such carriers, excipients and diluents include water, lactose, dextrose, fructose, sucrose, sorbitol, mannitol, polyethylene glycol, propylene glycol, starch, gum, gelatin, alginate, calcium silicate, calcium phosphate, cellulose, water syrup, methylcellulose, polyvinylpyrrolidone, alkyl parahydroxy benzosorbate, talc, magnesium stearate, stearic acid, glycerine, as well as various oils such as sesame oil, olive oil, soybean oil, and the like.

Moreover, the above carriers, excipients or diluents may optionally be supplemented with commonly used additives such as extenders, binders, disintegrating agents, pH adjustors, solubilizers and so on, and then formulated using standard techniques into oral or parenteral dosage forms including tablets, pills, capsules, granules, powders, solutions, emulsions, suspensions, ointments, injections, skin plasters, etc. The compounds of the present invention may be given to adult patients at 0.001 to 500 mg per administration, once or several times a day, by the oral or parenteral route. This dosage may be increased or decreased as appropriate for the type of disease to be treated, the age, body weight and symptom of a patient, etc.

Profiles desired for the compounds of the present invention include excellent efficacy, good in vivo kinetics, excellent physical properties, low toxicity, etc.

The compounds of the present invention can be prepared in the following manner.

(Process for Preparing the Compound of the Present Invention)

The compounds of the present invention can be prepared by known organic chemistry procedures, for example, according to the following reaction schemes.

In Reaction Schemes 1 to 7 shown below, R, R¹ to R⁷, T, n, A and B are as defined above. X¹ to X⁵, which may be the same or different, each represent a leaving group such as a halogen atom (e.g., a chlorine atom, a bromine atom, an iodine atom) or an organic sulfonyloxy group (e.g., a methanesulfonyloxy group, a benzenesulfonyloxy group, a p-toluenesulfonyloxy group, a trifluoromethanesulfonyloxy group), Y¹ and Y², which may be the same or different, each represent a leaving group (e.g., a halogen atom or an organic sulfonyloxy group) or a hydroxyl group, Z represents a carbon atom or an oxygen atom, p represents an integer of 0 or 1, and R⁸ and R⁹ each represent a hydrogen atom, an alkyl group or a cycloalkyl group. Alternatively, R⁸ and R⁹ may form a cycloalkyl ring together with their adjacent carbon atom.

Explanation will be given below of the process shown in Reaction Scheme 1 for preparing the compound of the present invention. This process is intended to prepare the compound (1) of the present invention from compound (2).

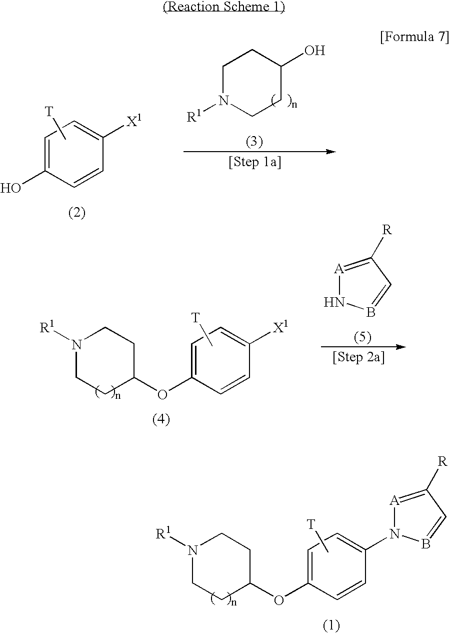

(Step 1a)

Step 1a is intended to obtain compound (4) by coupling reaction between compounds (2) and (3). Compounds (2) and (3) are known or may be easily synthesized from known compounds. Such coupling reaction may be Mitsunobu reaction, for example, which is accomplished in a solvent in the presence of a reagent composed of an organophosphorus compound (e.g., triphenylphosphine, tributylphosphine) in combination with an azo compound (e.g., diethyl azodicarboxylate, diisopropyl azodicarboxylate, di-tert-butyl azodicarboxylate) or in the presence of a phosphorus ylide reagent (e.g., cyanomethyltributylphosphorane). Examples of a solvent available for use in this reaction include ethers (e.g., tetrahydrofuran, 1,4-dioxane), hydrocarbons (e.g., toluene, benzene), halogenated hydrocarbons (e.g., chloroform, dichloromethane), amides (e.g., N,N-dimethylformamide, N,N-dimethylacetamide, N-methyl-2-pyrrolidone), dimethyl sulfoxide, acetonitrile, or mixed solvents thereof. Among them, preferred is tetrahydrofuran or toluene. The reaction temperature in this reaction generally ranges from 0° C. to 120° C., preferably from 15° C. to 80° C., and the reaction time generally ranges from 1 to 48 hours, preferably from 1 to 12 hours.

(Step 2a)

Step 2a is intended to obtain the compound (1) of the present invention by condensation between compounds (4) and (5) through coupling reaction. Compound (5) is known or may be easily synthesized from a known compound. Such coupling reaction may be accomplished by standard procedures used for aromatizing a nitrogen atom in an azole compound in the presence of a base by using a ligand and a catalyst in a solvent, for example, according to the method described in Kunz et al., Synlett, 15, 2428-2439, 2003 or equivalent methods thereof. Examples of a catalyst available for use in this reaction include copper catalysts commonly used for condensation reaction, as exemplified by copper(0), copper(I) iodide, copper(I) chloride, copper(I) oxide, copper(I) bromide tristriphenylphosphine complex, copper(I) trifluoromethanesulfonate benzene complex, etc. Examples of a ligand available for use in this reaction include ligands commonly used for condensation reaction using a copper catalyst, as exemplified by N,N′-dimethylethylenediamine, 1,2-cyclohexanediamine, 2-aminopyridine, 1,10-phenanthroline, 2-hydroxybenzaldehydeoxime, ethylene glycol, etc. Examples of a base available for use in this reaction include potassium carbonate, potassium phosphate, potassium hydroxide, potassium tert-butoxide, cesium carbonate, sodium carbonate, sodium bicarbonate, sodium acetate, sodium methoxide, and tetrabutylammonium hydroxide. Among them, preferred is potassium carbonate or cesium carbonate. Examples of a solvent available for use in this reaction include alcohols (e.g., methanol, ethanol, isopropanol), ethers (e.g., tetrahydrofuran, 1,4-dioxane), hydrocarbons (e.g., toluene, benzene), halogenated hydrocarbons (e.g., chloroform, dichloromethane), amides (e.g., N,N-dimethylformamide, N,N-dimethylacetamide, N-methyl-2-pyrrolidone), dimethyl sulfoxide, acetonitrile, acetone, water, or mixed solvents thereof. Among them, preferred is N,N-dimethylformamide or N-methyl-2-pyrrolidone. The reaction temperature in this reaction generally ranges from 0° C. to 150° C., preferably from 40° C. to 120° C., and the reaction time generally ranges from 1 to 48 hours, preferably from 1 to 12 hours.

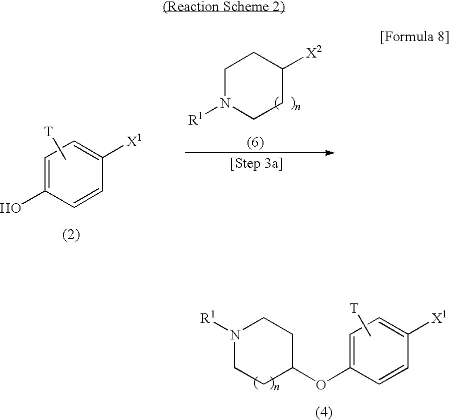

Compound (4) may also be obtained by coupling reaction between compounds (2) and (6). Compound (6) is known or may be easily synthesized from a known compound. Such coupling reaction may be accomplished by standard procedures for O-alkylation of phenol in the presence or absence of a base with or without a solvent. If necessary, for example, an additive such as potassium iodide or sodium bromide may be added. Examples of a base available for use in this reaction include pyridine, triethylamine, diisopropylethylamine, potassium tert-butoxide, potassium carbonate, cesium carbonate, sodium bicarbonate, sodium hydroxide, potassium hydroxide, and sodium hydride. Examples of a solvent available for use in this reaction include alcohols (e.g., methanol, ethanol, isopropanol), ethers (e.g., tetrahydrofuran, 1,4-dioxane), hydrocarbons (e.g., toluene, benzene), halogenated hydrocarbons (e.g., chloroform, dichloromethane), amides (e.g., N,N-dimethylformamide, N,N-dimethylacetamide, N-methyl-2-pyrrolidone), dimethyl sulfoxide, acetonitrile, acetone, water, or mixed solvents thereof. Among them, preferred is tetrahydrofuran, N-dimethylformamide, acetonitrile or acetone. The reaction temperature in this reaction generally ranges from 0° C. to 150° C., preferably from 15° C. to 80° C., and the reaction time generally ranges from 1 to 48 hours, preferably from 1 to 12 hours.

Explanation will be given below of the process shown in Reaction Scheme 3 for preparing the compound of the present invention. This process is intended to prepare the compound (1) of the present invention from compound (2).

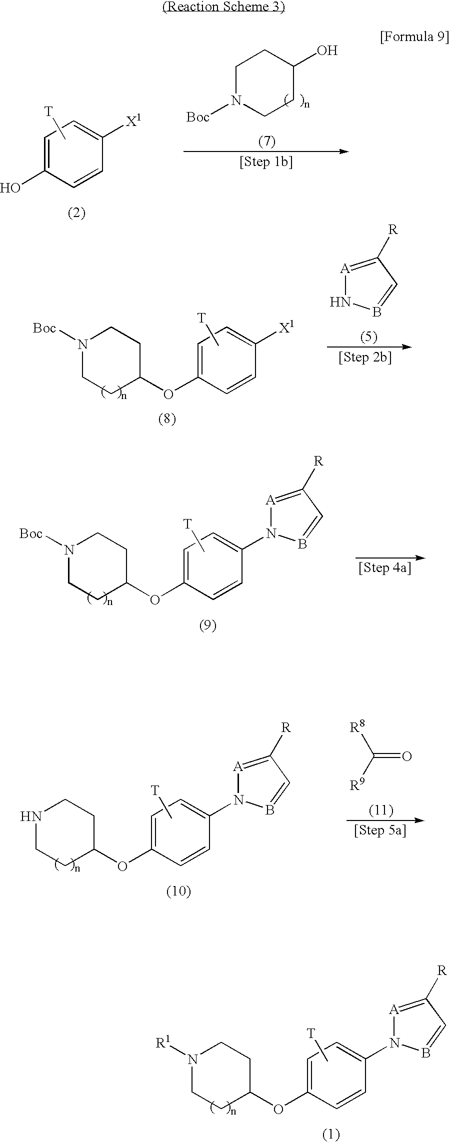

(Step 1b)

Step 1b is intended to obtain compound (8) by condensation between compound (2) and known compound (7) through coupling reaction. Such coupling reaction may be accomplished in the same manner as shown in Step 1a.

(Step 2b)

Step 2b is intended to obtain compound (9) by condensation between compounds (8) and (5) through coupling reaction. Such coupling reaction may be accomplished in the same manner as shown in Step 2a.

(Step 4a)

Step 4a is intended to obtain compound (10) by deprotection of the tert-butoxycarbonyl group in compound (9). Such reaction may be accomplished by standard procedures for deprotection of a tert-butoxycarbonyl group, for example, according to the method described in T. W. Greene and P. G. M. Wuts, Protective Groups in Organic Synthesis or equivalent methods thereof. By way of example, such reaction may be accomplished in the presence of a strong acid with or without a solvent, or in the presence of a base in a solvent. Examples of an acid available for use in this reaction include hydrochloric acid, sulfuric acid, trifluoroacetic acid, and trifluoromethanesulfonic acid. Examples of a base available for use in this reaction include sodium hydroxide and potassium hydroxide. Examples of a solvent available for use in this reaction include alcohols (e.g., methanol, ethanol, isopropanol), ethers (e.g., diethyl ether, tetrahydrofuran, 1,4-dioxane), hydrocarbons (e.g., toluene, benzene), halogenated hydrocarbons (e.g., chloroform, dichloromethane), amides (e.g., N,N-dimethylformamide, N,N-dimethylacetamide, N-methyl-2-pyrrolidone), dimethyl sulfoxide, acetonitrile, acetone, water, or mixed solvents thereof. The reaction temperature in this reaction generally ranges from 0° C. to 150° C., preferably from 15° C. to 40° C., and the reaction time generally ranges from 1 to 48 hours, preferably from 1 to 12 hours.

(Step 5a)

Step 5a is intended to obtain the compound (1) of the present invention by condensation between compounds (10) and (11) through coupling reaction. Compound (11) is known or may be easily synthesized from a known compound. Such coupling reaction may be accomplished by standard procedures for reductive amination through condensation between a carbonyl compound and an amine, for example, by adding a reducing agent to a mixture of compounds (8) and (9) in the presence or absence of an acid with or without a solvent. As another example, it is possible to use catalytic reduction through hydrogenation using a catalyst such as palladium-carbon, platinum, Raney Nickel or rhodium-alumina. Examples of a reducing agent available for use in this reaction include sodium borohydride, sodium triacetoxyborohydride, sodium cyanoborohydride, diborane, and lithium aluminum hydride. Examples of an acid available for use in this reaction include acetic acid, formic acid, and hydrochloric acid. Examples of a solvent available for use in this reaction include alcohols (e.g., methanol, ethanol, isopropanol), ethers (e.g., tetrahydrofuran, 1,4-dioxane), hydrocarbons (e.g., toluene, benzene), acetonitrile, water, or mixed solvents thereof. Among them, preferred is ethanol, toluene or tetrahydrofuran. The reaction temperature in this reaction generally ranges from 0° C. to 150° C., preferably from 15° C. to 40° C., and the reaction time generally ranges from 1 to 48 hours, preferably from 1 to 12 hours.

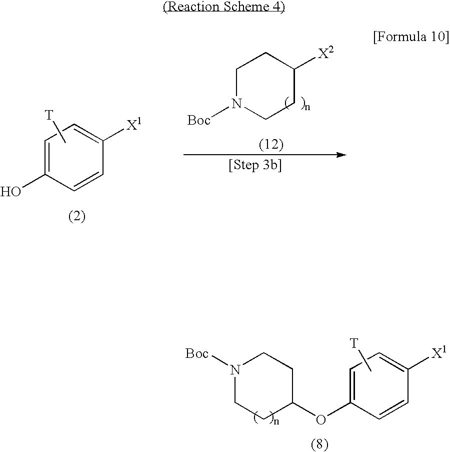

Compound (8) may also be obtained by condensation between compounds (2) and (12) through coupling reaction. Compound (12) is known or may be easily synthesized from a known compound. Such coupling reaction may be accomplished in the same manner as shown in Step 3a.

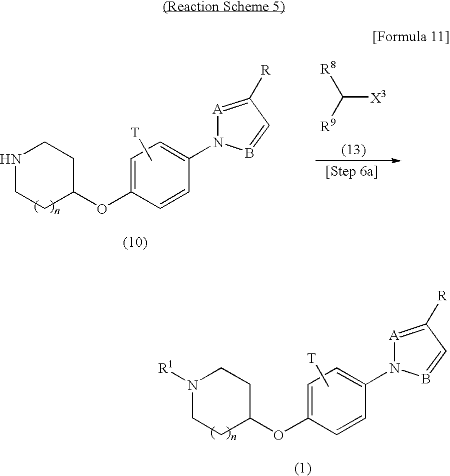

The compound (1) of the present invention may also be obtained by coupling reaction between compounds (10) and (13). Compound (13) is known or may be easily synthesized from a known compound. Such coupling reaction may be accomplished by standard procedures for alkylation of amines in the presence or absence of a base with or without a solvent. If necessary, for example, an additive such as potassium iodide or sodium bromide may be added. Examples of a base available for use in this reaction include pyridine, triethylamine, diisopropylethylamine, potassium tert-butoxide, potassium carbonate, cesium carbonate, sodium bicarbonate, sodium hydroxide, potassium hydroxide, and sodium hydride. Examples of a solvent available for use in this reaction include alcohols (e.g., methanol, ethanol, isopropanol), ethers (e.g., tetrahydrofuran, 1,4-dioxane), hydrocarbons (e.g., toluene, benzene), halogenated hydrocarbons (e.g., chloroform, dichloromethane), amides (e.g., N,N-dimethylformamide, N,N-dimethylacetamide, N-methyl-2-pyrrolidone), dimethyl sulfoxide, acetonitrile, acetone, water, or mixed solvents thereof. Among them, preferred is tetrahydrofuran, N,N-dimethylformamide or acetonitrile. The reaction temperature in this reaction generally ranges from 0° C. to 150° C., preferably from 15° C. to 80° C., and the reaction time generally ranges from 1 to 48 hours, preferably from 1 to 12 hours.

Explanation will be given below of the process shown in Reaction Scheme 6 for preparing the compound of the present invention. This process is intended to prepare the compounds (1-1) and (1-2) of the present invention from compounds (8) and (14).

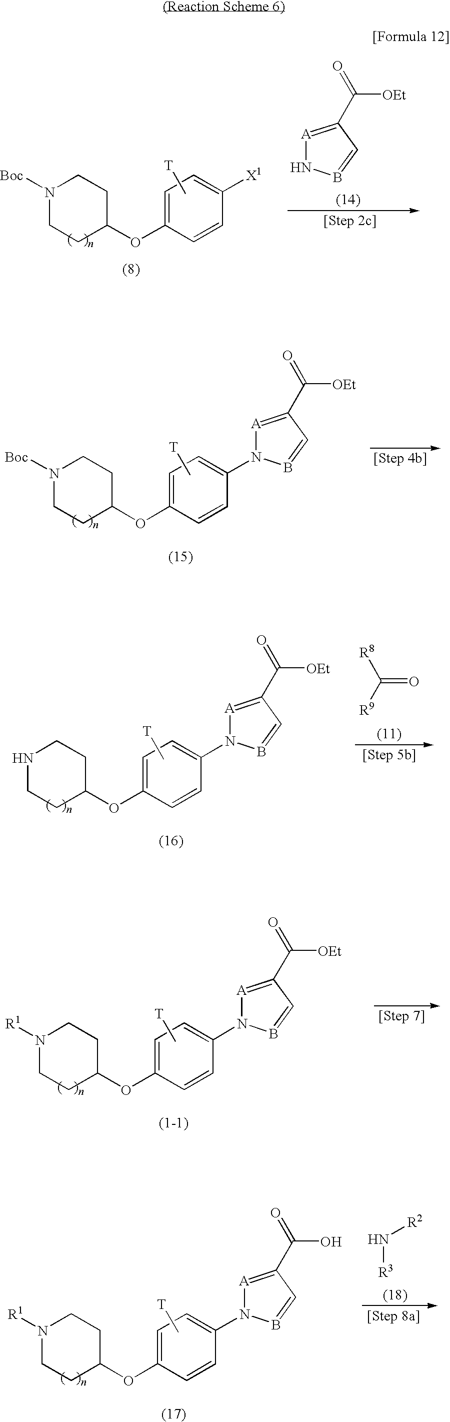

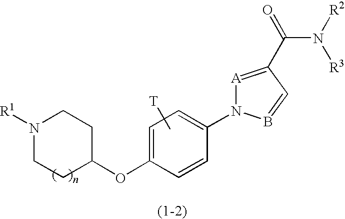

(Step 2c)

Step 2c is intended to obtain compound (15) by coupling reaction between compound (8) and known compound (14). Such coupling reaction may be accomplished in the same manner as shown in Step 2a.

(Step 4b)

Step 4b is intended to obtain compound (16) by removal of the tert-butoxycarbonyl group in compound (15). Such removal may be accomplished in the same manner as shown in Step 4a.

(Step 5b)

Step 5b is intended to obtain the compound (1-1) of the present invention by coupling reaction between compounds (16) and (11). Such coupling reaction may be accomplished in the same manner as shown in Step 5a.

(Step 7)

Step 7 is intended to obtain compound (17) by converting the ethoxycarbonyl group in compound (1-1) into a carboxylic acid through hydrolysis. Such hydrolysis may be accomplished by standard reaction for ester hydrolysis, for example, according to the method described in T. W. Greene and P. G. M. Wuts, Protective Groups in Organic Synthesis, third edition, John Wiley and Sons or equivalent methods thereof. By way of example, such reaction may be accomplished in the presence of a strong acid with or without a solvent, or in the presence of a base in a solvent. The reaction temperature in this reaction generally ranges from 0° C. to 120° C., preferably from 15° C. to 80° C., and the reaction time generally ranges from 1 to 48 hours, preferably from 1 to 12 hours.

(Step 8a)

Step 8a is intended to obtain the compound (1-2) of the present invention by condensation between compounds (17) and (18) through coupling reaction. Compound (18) is known or may be easily synthesized from a known compound. Such coupling reaction may be accomplished by standard procedures for amidation of carboxylic acids, for example, through conversion of a carboxylic acid into a carboxylic acid halide (e.g., carboxylic acid chloride, carboxylic acid bromide) and the subsequent reaction with an amine, through reaction of a mixed acid anhydride (e.g., obtained from a carboxylic acid and a chlorocarbonate ester) with an amine, through conversion of a carboxylic acid into an active ester (e.g., 1-benzotriazolyl ester, succinimidyl ester) and the subsequent reaction with an amine, or through reaction of a carboxylic acid with an amine in the presence of a dehydration condensing agent. All of these reactions may be accomplished in the presence or absence of a base in a solvent. Examples of a dehydration condensing agent available for use in this reaction include 3-(3-dimethylaminopropyl)-1-ethylcarbodiimide hydrochloride, dicyclohexylcarbodiimide, diphenylphosphorylazide, carbonyldiimidazole, and O-(7-azabenzotriazol-1-yl)-1,1,3,3-tetramethyluronium hexafluorophosphate. If necessary, it is possible to use an activator such as 1-hydroxybenzotriazole or hydroxysuccinimide. Examples of a base available for use in this reaction include pyridine, triethylamine, diisopropylethylamine, potassium carbonate, sodium carbonate, and sodium bicarbonate. Examples of a solvent available for use in this reaction include ethers (e.g., tetrahydrofuran, 1,4-dioxane), hydrocarbons (e.g., toluene, benzene), halogenated hydrocarbons (e.g., chloroform, dichloromethane), amides (e.g., N,N-dimethylformamide, N,N-dimethylacetamide, N-methyl-2-pyrrolidone), dimethyl sulfoxide, acetonitrile, acetone, water, or mixed solvents thereof. Among them, preferred is toluene, tetrahydrofuran or N,N-dimethylformamide. The reaction temperature in this reaction generally ranges from 0° C. to 120° C., preferably from 15° C. to 40° C., and the reaction time generally ranges from 1 to 48 hours, preferably from 1 to 12 hours.

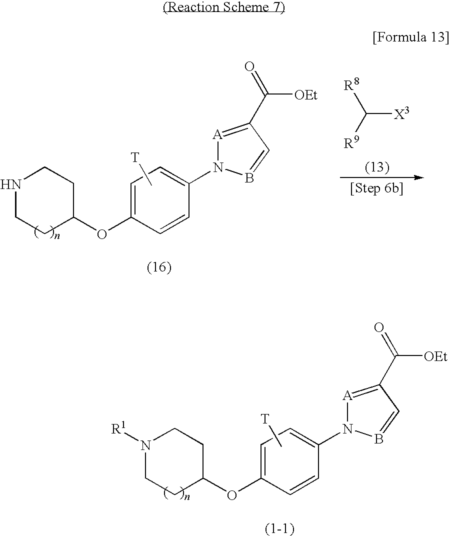

The compound (1-1) of the present invention may also be obtained by coupling reaction between compounds (16) and (13). Such coupling reaction may be accomplished in the same manner as shown in Step 6a.

Explanation will be given below of the process shown in Reaction Scheme 8 for preparing the compound of the present invention. This process is intended to prepare the compounds (1-3) and (1-4) of the present invention from compound (1-1).

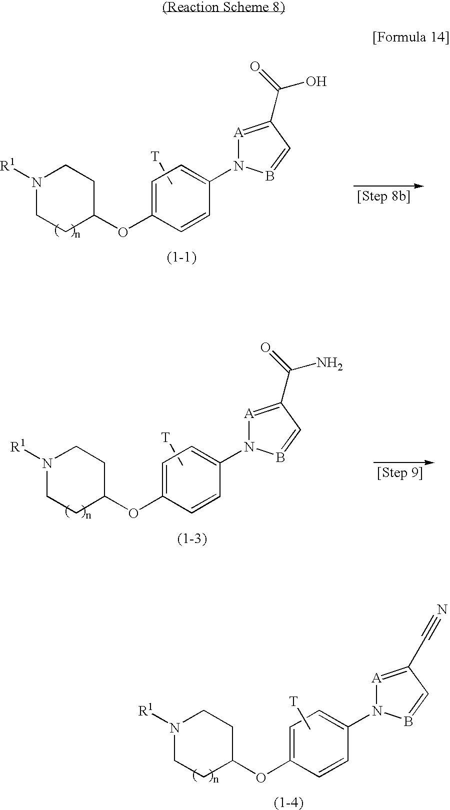

(Step 8b)

Step 8b is intended to obtain the compound (1-3) of the present invention by reaction between compound (1-1) and ammonia. Such reaction may be accomplished in the same manner as shown in Step 8a, more specifically by reacting the carboxylic acid with aqueous ammonia in the presence of a dehydration condensing agent, by way of example.

(Step 9)

Step 9 is intended to obtain the compound (1-4) of the present invention by converting the carbamoyl group in compound (1-3) into a nitrile group. This step may be accomplished by standard reaction for converting a carbamoyl group into a nitrile group, for example, in the presence of a dehydrating agent with or without a solvent. If necessary, for example, an additive such as N,N-dimethylformamide or sodium chloride may be added. Examples of a dehydrating agent available for use in this reaction include phosphorus pentaoxide, phosphorus pentachloride, phosphoryl chloride, thionyl chloride, oxalyl chloride, trifluoroacetic anhydride, and trifluoromethanesulfonic anhydride. When using a solvent in this reaction, examples of such a solvent include ethers (e.g., tetrahydrofuran, 1,4-dioxane), hydrocarbons (e.g., toluene, benzene), amides (e.g., N,N-dimethylformamide, N,N-dimethylacetamide, N-methyl-2-pyrrolidone), acetonitrile, or mixed solvents thereof. The reaction temperature in this reaction generally ranges from 0° C. to 120° C., preferably from 15° C. to 80° C., and the reaction time generally ranges from 1 to 48 hours, preferably from 1 to 12 hours.

Explanation will be given below of the process shown in Reaction Scheme 9 for preparing the compound of the present invention. This process is intended to prepare the compounds (1-5), (1-6) and (1-7) of the present invention from compound (4).

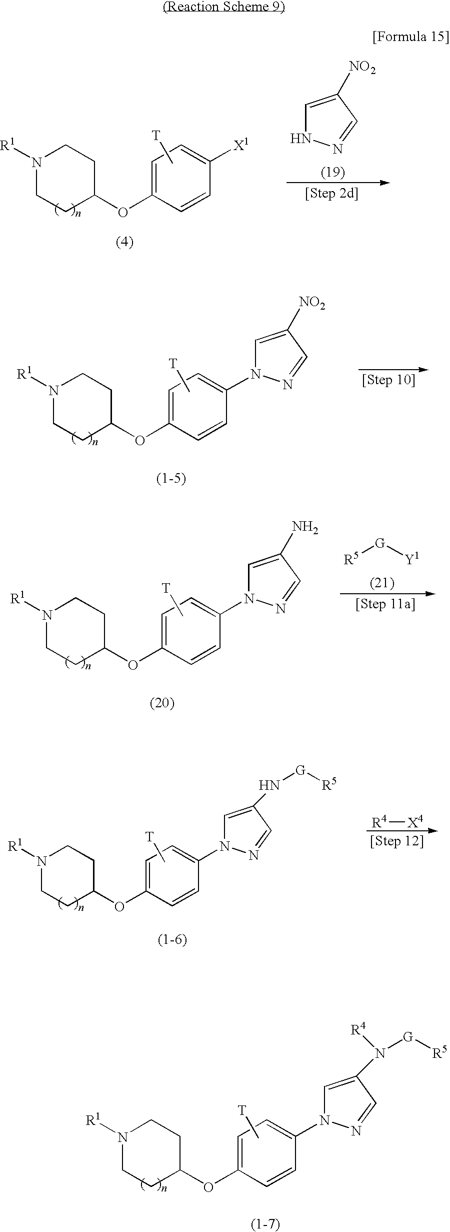

(Step 2d)

Step 2d is intended to obtain the compound (1-5) of the present invention by condensation between compounds (4) and (19) through coupling reaction. Such reaction may be accomplished in the same manner as shown in Step 2a.

(Step 10)

Step 10 is intended to obtain compound (20) by reducing the nitro group in compound (1-5). This step may be accomplished by standard reduction for converting a nitro group into an amino group, for example, by catalytic reduction through hydrogenation using a catalyst such as palladium-carbon, platinum, Raney Nickel or rhodium-alumina, by reduction with zinc, iron, tin or tin(II) chloride under acidic conditions, or by reduction with a metal hydride such as lithium aluminum hydride. More specifically, this step may be accomplished by catalytic reduction through hydrogenation using palladium-carbon as a catalyst in methanol solvent, by way of example.

(Step 11a)

Step 11a is intended to obtain the compound (1-6) of the present invention by condensation between compounds (20) and (21) through coupling reaction. Compound (21) is known or may be easily synthesized from a known compound. When G is CO and Y¹ is a hydroxyl group, such coupling reaction may be accomplished in the same manner as shown in Step 8a.

When Y¹ is a halogen atom, such coupling reaction may be accomplished by reacting compound (20) with compound (21) in the presence or absence of a base with or without a solvent. Examples of a base available for use in this reaction include pyridine, triethylamine, diisopropylethylamine, potassium carbonate, sodium bicarbonate, and sodium hydroxide. Examples of a solvent available for use in this reaction include ethers (e.g., tetrahydrofuran, 1,4-dioxane), hydrocarbons (e.g., toluene, benzene), halogenated hydrocarbons (e.g., chloroform, dichloromethane), amides (e.g., N,N-dimethylformamide, N,N-dimethylacetamide, N-methyl-2-pyrrolidone), or mixed solvents thereof. Among them, preferred is tetrahydrofuran or toluene. The reaction temperature in this reaction generally ranges from 0° C. to 120° C., preferably from 15° C. to 80° C., and the reaction time generally ranges from 1 to 48 hours, preferably from 1 to 12 hours.

(Step 12)

Step 12 is intended to obtain the compound (1-7) of the present invention by reaction between compounds (1-6) and (22). Compound (22) is known or may be easily synthesized from a known compound. Such reaction may be accomplished by standard procedures for alkylation of amides, for example, by reacting compound (1-6) with compound (22) in the presence of a base in a solvent. If necessary, for example, an additive such as tetrabutylammonium bromide or 18-crown-6-ether may be added. Examples of a base available for use in this reaction include sodium hydride, potassium hydride, potassium tert-butoxide, potassium hydroxide, sodium hydroxide, sodium methoxide, and n-butyllithium. Examples of a solvent available for use in this reaction include alcohols (e.g., methanol, ethanol, isopropanol), ethers (e.g., tetrahydrofuran, 1,4-dioxane), hydrocarbons (e.g., toluene, benzene), amides (e.g., N,N-dimethylformamide, N,N-dimethylacetamide, N-methyl-2-pyrrolidone), dimethyl sulfoxide, acetonitrile, water, or mixed solvents thereof. Among them, preferred is tetrahydrofuran or N,N-dimethylformamide. The reaction temperature in this reaction generally ranges from 0° C. to 150° C., preferably from 15° C. to 100° C., and the reaction time generally ranges from 1 to 48 hours, preferably from 1 to 12 hours.

Explanation will be given below of the process shown in Reaction Scheme 10 for preparing the compound of the present invention. This process is intended to prepare the compound (1-7) of the present invention from compound (20).

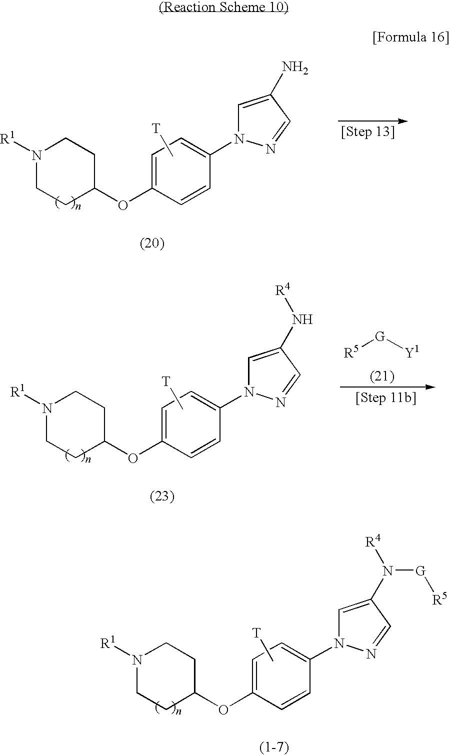

(Step 13)

Step 13 is intended to obtain compound (23) by alkylation of compound (20). Such alkylation may be accomplished by standard alkylation procedures for converting a primary amino group into a secondary amino group, for example, through reaction in the presence of a base using an alkylating agent (e.g., alkyl halide, alkyl methanesulfonate), through reductive amination with an aldehyde, through conversion into an acid amide using a carboxylic acid or a derivative thereof and the subsequent reduction with a metal hydride (e.g., borane), or through dehydration condensation with an alcohol.

(Step 11b)

Step 11b is intended to obtain the compound (1-7) of the present invention by condensation between compounds (23) and (21) through coupling reaction. Such coupling reaction may be accomplished in the same manner as shown in Step 11a.

Explanation will be given below of the process shown in Reaction Scheme 11 for preparing the compound of the present invention. This process is intended to prepare the compounds (1-8) and (1-9) of the present invention from compound (20).

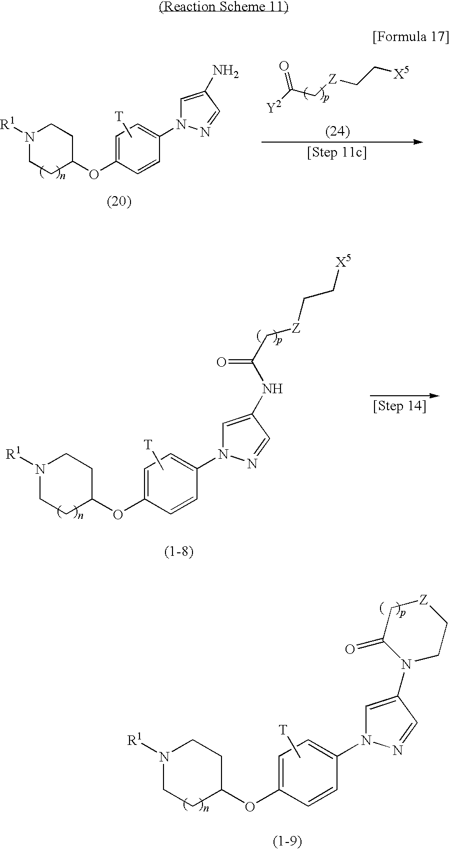

(Step 11c)

Step 11c is intended to obtain the compound (1-8) of the present invention by condensation between compounds (20) and (24) through coupling reaction. Compound (24) is known or may be easily synthesized from a known compound. Such coupling reaction may be accomplished in the same manner as shown in Step 11a.

(Step 14)

Step 14 is intended to obtain the compound (1-9) of the present invention by intramolecular cyclization of compound (1-8). Such intramolecular cyclization may be accomplished according to the methods described in documents (e.g., Journal of Medicinal Chemistry, 2002, vol. 45, pages 3972-3983) or equivalent methods thereof. Examples of a base available for use in this reaction include sodium hydride, potassium hydride, potassium carbonate, potassium tert-butoxide, and sodium hydroxide. Examples of a solvent available for use in this reaction include ethers (e.g., tetrahydrofuran, 1,4-dioxane), hydrocarbons (e.g., toluene, benzene), amides (e.g., N,N-dimethylformamide, N,N-dimethylacetamide, N-methyl-2-pyrrolidone), dimethyl sulfoxide, acetonitrile, acetone, or mixed solvents thereof. Among them, preferred is tetrahydrofuran or toluene. The reaction temperature in this reaction generally ranges from 0° C. to 150° C., preferably from 15° C. to 80° C., and the reaction time generally ranges from 1 to 48 hours, preferably from 1 to 12 hours.

The present invention will be further described in more detail by way of the following examples and test examples, which are not intended to limit the scope of the invention.

### Example 1

### Preparation of 1-cyclopentyl-4-{4-[4-(pyrrolidin-1-ylcarbonyl)-1H-pyrazol-1-yl]phenoxy}piperidine (Compound No. 1)

### (1) Preparation of tert-butyl 4-(4-iodophenoxy)piperidine-1-carboxylate

To a solution of tert-butyl 4-hydroxypiperidine-1-carboxylate (4.3 g), 4-iodophenol (4.8 g) and triphenylphosphine (5.8 g) in tetrahydrofuran (40 mL), 40% diisopropyl azodicarboxylate in toluene (11 mL) was added and stirred overnight at 60° C. The reaction mixture was cooled to room temperature and concentrated under reduced pressure. The resulting residue was purified by silica gel column chromatography (eluting solvent: n-hexane:ethyl acetate=20:1) to give the titled compound (5.8 g) as a colorless solid.

### (2) Preparation of tert-butyl 4-{-4-[4-(pyrrolidin-1-ylcarbonyl)-1H-pyrazol-1-yl]phenoxy}piperidine-1-carboxylate

A suspension of tert-butyl 4-(4-iodophenoxy)piperidine-1-carboxylate obtained in Example 1-(1) (1.5 g), 4-(pyrrolidin-1-ylcarbonyl)-1H-pyrazole (0.68 g), rac-trans-N,N′-dimethylcyclohexane-1,2-diamine (0.68 g), copper(I) iodide (0.085 g) and cesium carbonate (2.5 g) in N,N-dimethylformamide (2.5 mL) was stirred at 110° C. for 2 hours. The reaction mixture was cooled to room temperature, diluted with chloroform and filtered through celite. The filtrate was washed with water and concentrated under reduced pressure. The resulting residue was purified by silica gel column chromatography (eluting solvent: chloroform:methanol=9:1) and washed with ethyl acetate to give the titled compound (1.5 g) as a colorless solid.

### (3) Preparation of 4-{4-[4-(pyrrolidin-1-ylcarbonyl)-1H-pyrazol-1-yl]phenoxy}piperidine

To a solution of tert-butyl 4-{4-[4-(pyrrolidin-1-ylcarbonyl)-1H-pyrazol-1-yl]phenoxy}piperidine-1-carboxylate obtained in Example 1-(2) (1.5 g) in a mixed solvent of tetrahydrofuran (15 mL), methanol (5.0 mL) and chloroform (10 mL), 4M hydrochloric acid in ethyl acetate was added under ice cooling and stirred overnight at room temperature. The reaction mixture was diluted with saturated sodium bicarbonate and extracted with chloroform. The organic layer was dried over anhydrous magnesium sulfate and concentrated under reduced pressure. The resulting residue was washed with diisopropyl ether to give the titled compound (1.1 g) as a colorless solid.

### (4) Preparation of 1-cyclopentyl-4-{4-[4-(pyrrolidin-1-ylcarbonyl)-1-pyrazol-1-yl]phenoxy}piperidine (Compound No. 1)

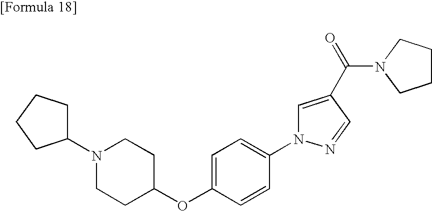

To a solution of 4-{4-[4-(pyrrolidin-1-ylcarbonyl)-1H-pyrazol-1-yl]phenoxy}piperidine obtained in Example 1-(3) (0.30 g), cyclopentanone (0.089 g) and acetic acid (0.079 g) in chloroform (3.0 mL), sodium triacetoxyborohydride (0.23 g) was added and stirred at room temperature for 1 hour. The reaction mixture was diluted with saturated aqueous sodium bicarbonate and extracted with chloroform. The organic layer was dried over anhydrous magnesium sulfate and concentrated under reduced pressure. The resulting residue was purified by silica gel column chromatography (eluting solvent: chloroform:methanol=8:1) and washed with diisopropyl ether to give the titled compound (0.28 g) as a colorless solid.

¹H NMR (600 MHz, CHLOROFORM-d) δ ppm 1.49-1.74 (m, 6H), 1.81-2.06 (m, 10H), 2.29-2.39 (m, 2H), 2.48-2.56 (m, 1H), 2.75-2.86 (m, 2H), 3.65 (t, J=6.9 Hz, 2H), 3.74 (t, J=6.6 Hz, 2H), 4.30-4.38 (m, 1H), 6.98 (d, J=9.2 Hz, 2H), 7.57 (d, J=9.2 Hz, 2H), 7.95 (s, 1H), 8.26 (s, 1H)

MS (ESI/APCI Dual) (Positive) m/z; 409 (M+H)⁺

Compounds (Compound Nos. 2 to 5) obtained in the same manner as shown in Example 1-(4) are summarized in Table 1.

TABLE 1

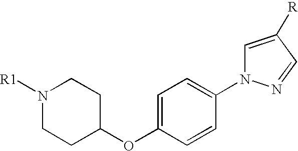

| | | | MS | |
|---|---|---|---|---|
| | | | (ESI/APCI | |
| Compound | | | Dual) | |
| No. | R1 | R | observe MH⁺ | ¹H NMR (600 MHz, CHLOROFORM-d) |
| 2 | 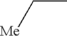 | 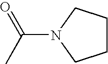 | m/z; 369 (M + H)⁺ | ¹H NMR (600 MHz, CHLOROFORM-d) δ ppm 1.10 (t, J = 7.1 Hz. 3 H), 1.80-1.89 (m, 2 H), 1.90-1.97 (m, 2 H), 1.98-2.09 (m, 4 H), 2.24-2.37 (m, 2 H), 2.44 (q, J = 7.1 Hz, 2 H), 2.67-2.81 (m, 2 H), 3.65 (t, J = 6.9 Hz, 2 H), 3.73 (t, J = 6.6 Hz, 2 H), 4.29-4.40 (m, 1 H), 6.98 (d, J = 9.2 Hz, 2 H), 7.57 (d, J = 9.2 Hz, 2 H), 7.94 (s, 1 H), 8.26 (s, 1 H) |
| 3 | 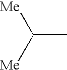 | 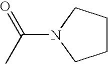 | m/z; 383 (M + H)⁺ | ¹H NMR (600 MHz, CHLOROFORM-d) δ ppm 1.06 (d, J = 6.4 Hz, 6 H), 1.77-2.08 (m, 8 H), 2.34-2.48 (m, 2 H), 2.71-2.84 (m, 3 H), 3.65 (t, J = 6.9 Hz, 2 H), 4.28-4.37 (m, 1 H), 6.98 (d, J = 8.7 Hz, 2 H), 7.57 (d, J = 9.2 Hz, 2 H), 7.94 (s, 1 H), 8.26 (s, 1 H) |
| 4 | 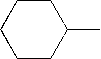 | 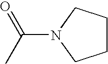 | m/z; 423 (M + H)⁺ | ¹H NMR (600 MHz, CHLOROFORM-d) δ ppm 1.04-1.14 (m, 1 H), 1.17-1.29 (m, 4 H), 1.58-2.06 (m, 13 H), 2.80-2.88 (m, 1 H), 3.65 (t, J = 6.9 Hz, 2 H), 3.73 (t, J = 6.6 Hz, 2 H), 4.27-4.35 (m, 1 H), 6.97 (d, J = 9.2 Hz, 2 H), 7.56 (d, J = 9.2 Hz, 2 H), 7.94 (s, 1 H), 8.26 (s, 1 H) |
| 5 | 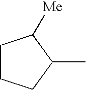 | 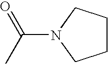 | m/z; 423 (M + H)⁺ | ¹H NMR (600 MHz, CHLOROFORM-d) δ ppm 0.84 (d, J = 7.3 Hz, 3 H), 1.38-1.52 (m, 2 H), 1.68-1.88 (m, 6 H), 1.91-2.07 (m, 6 H), 2.13-2.34 (m, 4 H), 2.78 (br. s., 2 H), 3.66 (t, J = 6.9 Hz, 2 H), 3.75 (t, J = 6.9 Hz, 2 H), 4.33 (br. s., 1 H), 6.99 (d, J = 9.2 Hz, 2 H), 7.58 (d, J = 8.7 Hz, 2 H), 7.96 (s, 1 H), 8.28 (s, 1 H) |

### Example 2

### Preparation of 1-(cyclopropylmethyl)-4-{4-[4-(pyrrolidin-1-ylcarbonyl)-1H-pyrazol-1-yl]phenoxy}piperidine (Compound No. 6)

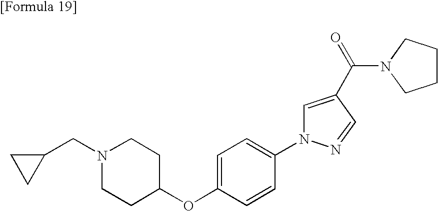

A suspension of 4-{4-[4-(pyrrolidin-1-ylcarbonyl)-1H-pyrazol-1-yl]phenoxy}piperidine obtained in Example 1-(3) (0.050 g), (bromomethyl)cyclopropane (0.024 g) and potassium carbonate (0.040 g) in N,N-dimethylformamide (0.50 mL) was stirred at 80° C. for 3 hours. The reaction mixture was cooled to room temperature, diluted with water and extracted with ethyl acetate. The organic layer was dried over anhydrous sodium sulfate and concentrated under reduced pressure. The resulting residue was purified by silica gel column chromatography (eluting solvent: chloroform:methanol=8:1) and washed with diisopropyl ether to give the titled compound (0.040 g) as a colorless solid.

¹H NMR (600 MHz, CHLOROFORM-d) δ ppm 0.12 (d, J=3.2 Hz, 2H), 0.54 (d, J=7.3 Hz, 2H), 0.89 (br. s, 1H), 1.84-2.09 (m, 8H), 2.25-2.45 (m, 4H), 2.86 (br. s, 2H), 3.66 (t, J=6.9 Hz, 2H), 3.75 (t, J=6.9 Hz, 2H), 4.36 (br. s, 1H), 6.99 (d, J=9.2 Hz, 2H), 7.58 (d, J=8.7 Hz, 2H), 7.96 (s, 1H), 8.28 (s, 1H)

MS (ESI/APCI Dual) (Positive) m/z; 395 (M+H)⁺

Compounds (Compound Nos. 7 to 11) obtained in the same manner as shown in Example 2 are summarized in Table 2.

TABLE 2

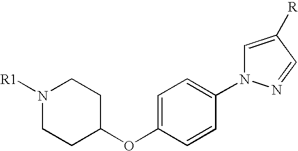

| | | | MS | |
|---|---|---|---|---|
| COm- | | | (ESI/APCI | |
| pound | | | Dual) | |
| No. | R1 | R | observe MH⁺ | ¹H NMR (600 MHz, CHLOROFORM-d) |
| 7 | 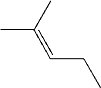 | 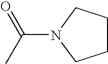 | m/z; 409 (M + H)⁺ | 1H NMR (600 MHz, CHLOROFORM-d) δ ppm 1.65 (s, 3 H), 1.73 (s, 3 H), 1.80-1.89 (m, 2 H), 1.90-2.06 (m, 6 H), 2.31 (br. s., 2 H), 2.73 (br. s., 2 H), 2.97 (d, J = 7.3 Hz, 2 H), 3.65 (t, J = 6.9 Hz, 2 H), 3.74 (t, J = 6.6 Hz, 2 H), 4.34 (br. s., 1 H), 5.27 (t, J = 7.1 Hz, 1 H), 6.95-7.00 (m, 2 H), 7.55-7.60 (m, 2 H), 7.94 (s, 1 H), 8.26 (s, 1 H) |
| 8 | 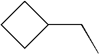 | 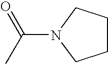 | m/z; 409 (M + H)⁺ | 1H NMR (600 MHz, CHLOROFORM-d) δ ppm 1.55 (br. s., 2 H), 1.63-1.74 (m, 3 H), 1.75-2.11 (m, 9 H), 2.22-2.29 (m, 2 H), 2.40-2.44 (m, 2 H), 2.49-2.58 (m, 1 H), 2.69 (br. s., 2 H), 3.65 (t, J = 6.9 Hz, 2 H), 3.74 (t, J = 6.7 Hz, 2 H), 4.31 (br. s., 1 H), 6.95-6.99 (m, 2 H), 7.55-7.58 (m, 2 H), 7.94 (s, 1 H), 8.26 (s, 1 H) |
| 9 | 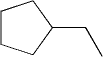 | 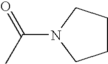 | m/z; 423 (M + H)⁺ | 1H NMR (600 MHz, CHLOROFORM-d) δ ppm 1.14-1.23 (m, 2 H), 1.46-1.63 (m, 4 H), 1.71-1.85 (m, 4 H), 1.90-2.09 (m, 7 H), 2.22-2.31 (m, 4 H), 2.74 (br. s., 2 H), 3.65 (t, J = 6.9 Hz, 2 H), 3.74 (t, J = 6.6 Hz, 2 H), 4.28-4.34 (m, 1 H), 6.95-7.00 (m, 2 H), 7.54-7.59 (m, 2 H), 7.94 (s, 1 H), 8.26 (s, 1 H) |
| 10 | 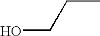 | 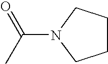 | m/z; 385 (M + H)⁺ | 1H NMR (600 MHz, CHLOROFORM-d) δ ppm 1.79-1.89 (m, 2 H), 1.90-2.07 (m, 6 H), 2.35-2.46 (m, 2 H), 2.52-2.61 (m, 2 H), 2.72-2.85 (m, 3 H), 3.61 (t, J = 15.3 Hz, 2 H), 3.65 (t, J = 6.9 Hz, 2 H), 3.74 (t, J = 6.9 Hz, 2 H), 4.33-4.42 (m, 1 H), 6.93-7.02 (m, 2 H), 7.54-7.62 (m, 2 H), 7.94 (s, 1 H), 8.27 (s, 1 H) |
| 11 | 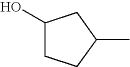 | 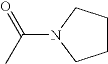 | m/z; 425 (M + H)⁺ | 1H NMR (600 MHz, CHLOROFORM-d) δ ppm 1.68-2.11 (m, 14 H), 2.38 (br. s., 2 H), 2.63-2.71 (m, 1 H), 2.80 (br. s., 2 H), 3.65 (t, J = 6.9 Hz, 2 H), 3.74 (t, J = 6.6 Hz, 3 H), 4.20-4.29 (m, 1 H), 4.35 (br. s., 1 H), 6.93-7.00 (m, 2 H), 7.54-7.60 (m, 2 H), 7.94 (s, 1 H), 8.27 (s, 1 H) |

### Example 3

### Preparation of ethyl 1-{4-[(1-cyclobutylpiperidin-4-yl)oxy]phenyl}-1H-pyrazole-4-carboxylate (Compound No. 12)

### (1) Preparation of tert-butyl 4-{-4-[4-(ethoxycarbonyl)-1H-pyrazol-1-yl]phenoxy}piperidine-1-carboxylate

A suspension of tert-butyl 4-(4-iodophenoxy)piperidine-1-carboxylate obtained in Example 1-(1) (4.0 g), ethyl 1H-pyrazole-4-carboxylate (1.5 g), rac-trans-N,N′-dimethylcyclohexane-1,2-diamine (0.79 g), copper(I) iodide (0.23 g) and cesium carbonate (6.8 g) in N,N-dimethylformamide (6.5 mL) was stirred at 110° C. for 3.5 hours. The reaction mixture was cooled to room temperature, diluted with chloroform and filtered through celite. The filtrate was washed with water, dried over anhydrous sodium sulfate and concentrated under reduced pressure. The resulting residue was washed with diisopropyl ether to give the titled compound (2.6 g) as a light-blue solid.

### (2) Preparation of ethyl 1-[4-(piperidin-4-yloxy)phenyl]-1H-pyrazole-4-carboxylate

To a solution of tert-butyl 4-{-4-[4-(ethoxycarbonyl)-1H-pyrazol-1-yl]phenoxy}piperidine-1-carboxylate obtained in Example 3-(1) (2.6 g) in chloroform (10 mL), 4M hydrochloric acid in ethyl acetate (10 mL) was added under ice cooling and stirred at room temperature for 2 hours. The reaction mixture was diluted with chloroform, and saturated aqueous sodium bicarbonate was added thereto, followed by extraction with chloroform. The organic layer was dried over anhydrous sodium sulfate and concentrated under reduced pressure. The resulting residue was washed with diisopropyl ether to give the titled compound (1.6 g) as a light-blue solid.

### (3) Preparation of ethyl 1-{4-[(1-cyclobutylpiperidin-4-yl)oxy]phenyl}-1H-pyrazole-4-carboxylate (Compound No. 12)

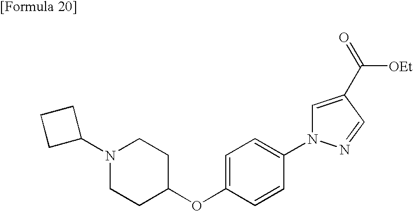

To a solution of ethyl 1-[4-(piperidin-4-yloxy)phenyl]-1H-pyrazole-4-carboxylate obtained in Example 3-(2) (1.6 g), cyclobutanone (0.42 g) and acetic acid (0.45 g) in chloroform (15 mL), sodium triacetoxyborohydride (1.3 g) was added and stirred overnight at room temperature. The reaction mixture was concentrated under reduced pressure, diluted with saturated aqueous sodium bicarbonate and extracted with chloroform. The organic layer was dried over anhydrous sodium sulfate and concentrated under reduced pressure. The resulting residue was washed with diisopropyl ether to give the titled compound (1.4 g) as a light-brown solid.

¹H NMR (600 MHz, CHLOROFORM-d) δ ppm 1.36 (t, J=7.1 Hz, 3H), 1.63-1.95 (m, 2H), 1.78-1.93 (m, 4H), 1.94-2.07 (m, 4H), 2.16 (br.s, 2H), 2.60 (br.s, 2H), 2.69-2.78 (m, 1H), 4.27-4.37 (m, 1H), 4.32 (q, J=7.2 Hz, 2H), 6.97 (d, J=9.2 Hz, 2H), 7.56 (d, J=9.2 Hz, 2H), 8.05 (s, 1H), 8.28 (s, 1H)

MS (ESI/APCI Dual) (Positive) m/z; 370 (M+H)⁺

### Example 4

### Preparation of ethyl 1-{4-[(1-cyclopentylpiperidin-4-yl)oxy]phenyl}-1H-pyrazole-4-carboxylate (Compound No. 13)

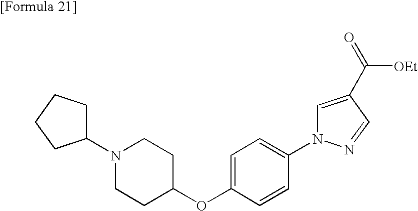

The same procedure as shown in Example 3-(3) was repeated to give the titled compound, except that cyclobutanone was replaced with cyclopentanone.

¹H NMR (600 MHz, CHLOROFORM-d) δ ppm 1.36 (t, J=7.1 Hz, 3H), 1.35-1.45 (m, 2H), 1.49-1.73 (m, 4H), 1.80-1.90 (m, 4H), 1.98-2.05 (m, 2H), 2.33 (br.s, 2H), 2.47-2.56 (m, 1H), 2.81 (br.s, 2H), 4.28-4.38 (m, 1H), 4.32 (q, J=7.3 Hz, 2H), 6.98 (d, J=9.2 Hz, 2H), 7.57 (d, J=9.2 Hz, 2H), 8.06 (s, 1H), 8.29 (s, 1H)

MS (ESI/APCI Dual) (Positive) m/z; 384 (M+H)⁺

### Example 5

### Preparation of 1-cyclobutyl-4-{4-[4-(pyrrolidin-1-ylcarbonyl)-1-pyrazol-1-yl]phenoxy}piperidine (Compound No. 14)

### (1) Preparation of 1-{4-[(1-cyclobutylpiperidin-4-yl)oxy]phenyl}-1H-pyrazole-4-carboxylic acid

A solution of ethyl 1-{4-[(1-cyclobutylpiperidin-4-yl)oxy]phenyl}-1H-pyrazole-4-carboxylate obtained in Example 3-(3) (1.4 g) and lithium hydroxide (0.27 g) in a mixed solvent of ethanol (7.0 mL) and water (7.0 mL) was stirred at 90° C. for 1 hour. The reaction mixture was cooled to room temperature and evaporated under reduced pressure to remove ethanol. The resulting residue was neutralized with 1M aqueous hydrochloric acid, diluted with water and stirred for 30 minutes under ice cooling. The precipitated crystal was collected by filtration to give the titled compound (1.2 g) as a colorless solid.

### (2) Preparation of 1-cyclobutyl-4-{4-[4-(pyrrolidin-1-ylcarbonyl)-1-pyrazol-1-yl]phenoxy}piperidine (Compound No. 14)

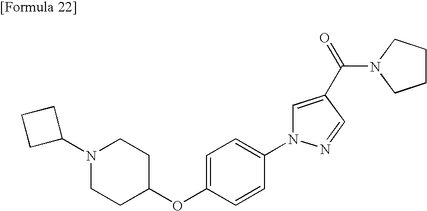

1-{4-[(1-Cyclobutylpiperidin-4-yl)oxy]phenyl}-1H-pyrazole-4-carboxylic acid obtained in Example 5-(1) (0.40 g), 1-{3-(dimethylamino)propyl}-3-ethylcarbodiimide hydrochloride (0.25 g), 1-hydroxybenzotriazole hydrate (0.20 g) and pyrrolidine (0.092 g) were added to N,N-dimethylformamide (4.0 mL) to give a suspension, which was then stirred overnight at room temperature. The reaction mixture was diluted with aqueous sodium bicarbonate and stirred at room temperature for 10 minutes. The precipitated crystal was filtered and dried, followed by washing with ethanol to give the titled compound (0.28 g) as a light-brown solid.

¹H NMR (600 MHz, CHLOROFORM-d) δ ppm 1.61-2.08 (m, 14H), 2.10-2.24 (m, 2H), 2.55-2.68 (m, 2H), 2.70-2.78 (m, 1H), 3.65 (t, J=6.9 Hz, 2H), 3.73 (t, J=6.6 Hz, 2H), 4.29-4.38 (m, 3H), 6.97 (d, J=9.2 Hz, 2H), 7.57 (d, J=8.7 Hz, 2H), 7.94 (s, 1H), 8.26 (s, 1H)

MS (ESI/APCI Dual) (Positive) m/z; 395 (M+H)⁺

### Example 6

### Preparation of 1-[(1-{4-[(1-cyclopentylpiperidin-4-yl)oxy]phenyl}-1H-pyrazol-4-yl)carbonyl]pyrrolidin-3-ol (Compound No. 15)

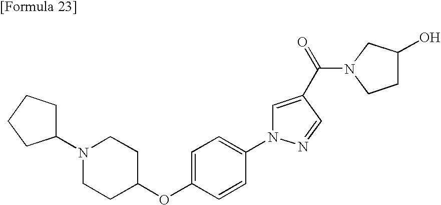

The same procedure as shown in Example 5 was repeated to give the titled compound, except that ethyl 1-{4-[(1-cyclobutylpiperidin-4-yl)oxy]phenyl}-1H-pyrazole-4-carboxylate was replaced with ethyl 1-{-4-[(1-cyclopentylpiperidin-4-yl)oxy]phenyl}-1H-pyrazole-4-carboxylate obtained in Example 4, and pyrrolidine was replaced with 3-hydroxypyrrolidine.

¹H NMR (600 MHz, CHLOROFORM-d) δ ppm 1.38-1.45 (m, 2H), 1.49-1.64 (m, 2H), 1.65-1.74 (m, 2H), 1.81-2.15 (m, 9H), 2.34 (br. s, 2H), 2.48-2.56 (m, 1H), 2.80 (br. s, 2H), 3.70-4.00 (m, 4H), 4.31-4.37 (m, 1H), 4.55-4.64 (m, 1H), 6.98 (d, J=8.7 Hz, 2H), 7.56 (d, J=8.7 Hz, 2H), 7.90-7.99 (m, 1H), 8.23-8.29 (m, 1H)

MS (ESI/APCI Dual) (Positive) m/z; 425 (M+H)⁺

Compounds (Compound Nos. 16 to 48) obtained in the same manner as shown in Example 5 or 6 are summarized in Tables 3-1 to 3-7.

TABLE 3-1

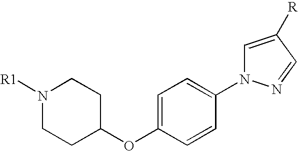

| | | | MS | |
|---|---|---|---|---|
| Com- | | | (ESI/APCI | |
| pound | | | Dual) | |
| No. | R1 | R | observe MH⁺ | ¹H NMR (600 MHz, CHLOROFORM-d) |
| 16 | 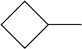 | 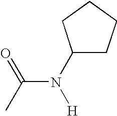 | m/z; 409 (M + H)⁺ | ¹H NMR (600 MHz, CHLOROFORM-d) δ ppm 1.42-2.22 (m, 20 H), 2.55-2.79 (m, 3 H), 4.29-4.43 (m, 2 H), 5.70 (d, J = 6.9 Hz, 1 H), 6.97 (d, J = 9.2 Hz, 2 H), 7.55 (d, J = 9.2 Hz, 2 H), 7.84 (s, 1 H), 8.24 (s, 1 H) |
| 17 | 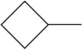 | 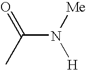 | m/z; 355 (M + H)⁺ | ¹H NMR (600 MHz, CHLOROFORM-d) δ ppm 1.63-2.25 (m, 12 H), 2.55-2.79 (m, 3 H), 3.00 (d, J = 5.0 Hz, 3 H), 5.79-5.83 (m, 1 H), 6.98 (d, J = 9.2 Hz, 2 H), 7.57 (d, J = 9.2 Hz, 2 H), 7.86 (s, 1 H), 8.27 (s, 1 H) |
| 18 | 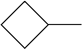 | 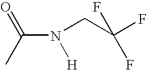 | m/z; 423 (M + H)⁺ | ¹H NMR (600 MHz, CHLOROFORM-d) δ ppm 1.65-1.76 (m, 2 H), 1.80-1.94 (m, 4 H), 1.98-2.09 (m, 4 H), 2.17 (br. s., 2 H), 2.57-2.68 (m, 2 H), 2.71-2.78 (m, 1 H), 4.08-4.15 (m, 2 H), 4.36 (br. s., 1 H), 6.03 (t, J = 6.6 Hz, 1 H), 6.99 (d, J = 8.7 Hz, 2 H), 7.57 (d, J = 9.2 Hz, 2 H), 7.94 (s, 1 H), 8.32 (s, 1 H) |
| 19 | 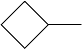 | 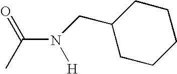 | m/z; 437 (M + H)⁺ | ¹H NMR (600 MHz, CHLOROFORM-d) δ ppm 0.96-1.04 (m, 2 H), 1.12-1.30 (m, 4 H), 1.63-1.93 (m, 11 H), 1.97-2.08 (m, 4 H), 2.17 (br. s., 2 H), 2.63 (br. s., 2 H), 2.71-2.79 (m, 1 H), 3.28 (t, J = 6.4 Hz, 2 H), 4.36 (br. s., 1 H), 5.84 (t, J = 6.0 Hz, 1 H), 6.98 (d, J = 9.2 Hz, 2 H), 7.56 (d, J = 8.7 Hz, 2 H), 7.87 (s, 1 H), 8.26 (d, J = 0.9 Hz, 1 H) |

TABLE 3-2

| | | | MS | |
|---|---|---|---|---|
| | | | (ESI/APCI | |
| Compound | | | Dual) | |
| No. | R1 | R | observe MH⁺ | ¹H NMR (600 MHz, CHLOROFORM-d) |
| 20 | 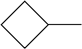 | 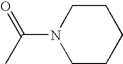 | m/z; 409 (M + H)⁺ | ¹H NMR (600 MHz, CHLOROFORM-d) δ ppm 1.50-2.25 (m, 18 H) 2.55-2.81 (m, 3 H) 3.62-3.71 (m, 4 H) 4.27-4.41 (m, 1 H) 6.94-7.00 (m, 2 H) 7.52-7.61 (m, 2 H) 7.77 (s, 1 H) 8.11 (s, 1 H) |
| 21 | 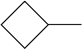 | 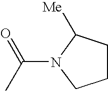 | m/z; 409 (M + H)⁺ | ¹H NMR (600 MHz, CHLOROFORM-d) δ ppm 1.22-1.37 (m, 3 H), 1.56 (s, 3 H), 1.58-2.25 (m, 13 H), 2.53-2.80 (m, 3 H), 3.66-3.82 (m, 2 H), 4.29-4.42 (m, 2 H), 6.97 (d, J = 8.7 Hz, 2 H), 7.56 (d, J = 8.7 Hz, 2 H), 7.93 (s, 1 H), 8.25 (s, 1 H) |
| 22 | 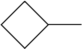 | 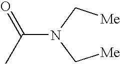 | m/z; 397 (M + H)⁺ | 1H NMR (600 MHz, CHLOROFORM-d) δ ppm 1.26 (br. s., 6 H), 1.57 (s, 3 H), 1.62-2.23 (m, 11 H), 2.58-2.78 (m, 3 H), 3.49-3.59 (m, 4 H), 4.34 (br. s., 1 H), 6.95-7.00 (m, 2 H), 7.53-7.58 (m, 2 H) |
| 23 | 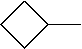 | 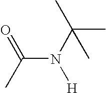 | m/z; 397 (M + H)⁺ | 1H NMR (600 MHz, CHLOROFORM-d) δ ppm 1.47 (s, 9 H), 1.62-2.21 (m, 12 H), 2.55-2.79 (m, 3 H), 4.35 (br.s, 1 H), 5.61 (br.s, 1 H), 6.98 (d, J = 9.2 Hz, 2 H), 7.55 (d, J = 9.2 Hz, 2 H), 7.81 (s, 1 H), 8.21 (s, 1 H) |
| 24 | 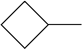 | 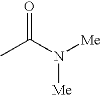 | m/z; 369 (M + H)⁺ | 1H NMR (600 MHz, CHLOROFORM-d) δ ppm 1.63-1.76 (m, 2 H), 1.79-1.94 (m, 4 H), 1.96-2.09 (m, 4 H), 2.17 (br. s., 2 H), 2.63 (br. s., 2 H), 2.71-2.79 (m, 1 H), 3.03-3.34 (m, 6 H), 4.35 (br. s., 1 H), 6.96-7.00 (m, 2 H), 7.54-7.59 (m, 2 H), 7.86 (s, 1 H), 8.18 (s, 1 H) |

TABLE 3-3

| | | | | |
|---|---|---|---|---|
| | | | MS | |
| | | | (ESI/APCI | |
| Compound | | | Dual) | |
| No. | R1 | R | observe MH⁺ | ¹H NMR (600 MHz, CHLOROFORM-d) |

| | | | | |
|---|---|---|---|---|
| 25 | 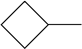 | 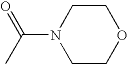 | m/z; 411 (M + H)⁺ | 1H NMR (600 MHz, CHLOROFORM-d) δ ppm 1.63-1.75 (m, 2 H), 1.80-1.94 (m, 4 H), 1.97-2.08 (m, 4 H), 2.17 (br. s., 2 H), 2.63 (br. s., 2 H), 2.71-2.79 (m, 1 H), 3.70-3.80 (m, 8 H), 4.35 (br. s., 1 H), 6.96-7.00 (m, 2 H), 7.54-7.57 (m, 2 H), 7.78 (s, 1 H), 8.14 (s, 1 H) |
| 26 | 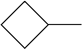 | 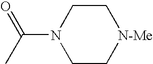 | m/z; 424 (M + H)⁺ | 1H NMR (600 MHz, CHLOROFORM-d) δ ppm 1.63-1.76 (m, 2 H), 1.79-1.95 (m, 4 H), 1.95-2.09 (m, 4 H), 2.17 (br. s., 2 H), 2.34 (s, 3 H), 2.46 (br. s., 4 H), 2.63 (br. s., 2 H), 2.70-2.79 (m, 1 H), 3.71-3.83 (m, 4 H), 4.35 (br. s., 1 H), 6.98 (d, J = 9.2 Hz, 2 H), 7.56 (d, J = 9.2 Hz, 2 H), 7.78 (s, 1 H), 8.12 (s, 1 H) |
| 27 | 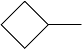 | 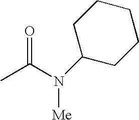 | m/z; 437 (M + H)⁺ | 1H NMR (600 MHz, CHLOROFORM-d) δ ppm 1.08-1.17 (m, 1 H), 1.22-1.95 (m, 15 H), 1.97-2.09 (m, 4 H), 2.17 (br. s., 2 H), 2.63 (br. s., 2 H), 2.70-2.79 (m, 1 H), 2.89-3.17 (m, 3 H), 3.88-4.60 (m, 2 H), 6.98 (d, J = 9.2 Hz, 2 H), 7.57 (d, J = 8.7 Hz. 2 H), 7.67-7.94 (m, 1 H), 8.16 (s, 1 H) |
| 28 | 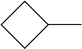 | 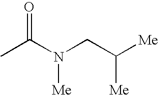 | m/z; 411 (M + H)⁺ | 1H NMR (600 MHz, CHLOROFORM-d) δ ppm 0.83-1.03 (m, 6 H), 1.63-1.75 (m, 2H), 1.79-1.94 (m, 4 H), 1.96-2.11 (m, 5 H), 2.17 (br. s., 2 H), 2.55-2.70 (m, 2 H), 2.71-2.78 (m, 1 H), 3.01-3.31 (m, 3 H), 3.38 (d, J = 7.8 Hz, 2 H), 4.35 (br. s., 1 H), 6.98 (d, J = 8.7 Hz, 2 H), 7.57 (d, J = 8.7 Hz, 2 H), 7.75-7.93 (m, 1 H), 8.09-8.24 (m, 1 H) |
| 29 | 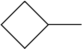 | 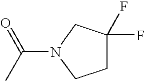 | m/z; 431 (M + H)⁺ | 1H NMR (600 MHz, CHLOROFORM-d) δ ppm 1.63-1.76 (m, 2 H), 1.78-1.84 (m, 4 H), 1.95-2.08 (m, 4 H), 2.16 (br. s., 2 H), 2.35-2.68 (m, 4 H), 2.69-2.78 (m, 1 H), 3.84-4.14 (m, 4 H), 4.34 (br. s., 1 H), 6.94-7.02 (m, 2 H), 7.51-7.61 (m, 2 H), 7.82-8.01 (m, 1 H), 8.25 (br. s., 1 H) |

TABLE 3-4

| | | | MS | |
|---|---|---|---|---|
| Com- | | | (ESI/APCI | |
| pound | | | Dual) | |
| No. | R1 | R | observe MH⁺ | ¹H NMR (600 MHz, CHLOROFORM-d) |
| 30 | 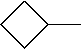 | 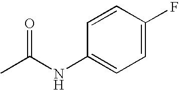 | m/z; 435 (M + H)⁺ | 1H NMR (600 MHz, CHLOROFORM-d) d ppm 1.64-2.28 (m, 12 H), 2.57-2.85 (m, 3 H), 4.38 (br. s., 1 H), 7.00 (d, J = 9.2 Hz, 2 H), 7.07 (t, J = 8.7 Hz, 2 H), 7.54-7.65 (m, 4 H), 8.00 (br. s., 1 H), 8.37 (s, 1 H) |
| 31 | 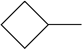 | 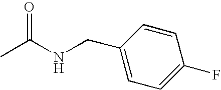 | m/z; 449 (M + H)⁺ | 1H NMR (600 MHz, CHLOROFORM-d) d ppm 1.64-1.75 (m, 2 H), 1.80-1.93 (m, 4 H), 1.98-2.08 (m, 4 H), 2.17 (br. s., 2 H), 2.62 (br. s., 2 H), 2.71-2.78 (m, 1 H), 4.35 (br. s., 1 H), 4.60 (d, J = 5.5 Hz, 2 H), 6.08 (t, J = 5.7 Hz, 1 H), 6.98 (d, J = 8.7 Hz, 2 H), 7.04 (t, J = 8.7 Hz, 2 H), 7.33 (dd, J = 8.7, 5.5 Hz, 2 H), 7.56 (d, J = 9.2 Hz, 2 H), 7.88 (s, 1 H), 8.29 (s, 1 H) |
| 32 | 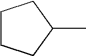 | 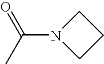 | m/z; 395 (M + H)⁺ | 1H NMR (600 MHz, CHLOROFORM-d) δ ppm 1.36-1.45 (m, 2 H), 1.50-1.61 (m, 2 H), 1.65-1.73 (m, 2 H), 1.81-1.91 (m, 4 H), 1.99-2.05 (m, 2 H), 2.29-2.43 (m, 4 H), 2.48-2.55 (m, 1 H), 2.80 (br. s., 2 H), 4.17-4.24 (m, 2 H), 4.30-4.36 (m, 1 H), 4.41-4.47 (m, 2 H), 6.95-7.00 (m, 2 H), 7.54-7.57 (m, 2 H), 7.85 (s, 1 H), 8.22 (s, 1 H) |
| 33 | 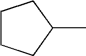 | 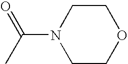 | m/z; 425 (M + H)⁺ | 1H NMR (600 MHz, CHLOROFORM-d) δ ppm 1.36-1.47 (m, 2 H), 1.50-1.61 (m, 2 H), 1.65-1.74 (m, 2 H), 1.81-1.91 (m, 4 H), 1.98-2.05 (m, 2 H), 2.28-2.39 (m, 2 H), 2.47-2.56 (m, 1 H), 2.76-2.85 (m, 2 H), 3.68-3.79 (m, 8 H), 4.30-4.37 (m, 1 H), 6.98 (m, 2 H), 7.52-7.57 (m, 2 H), 7.77 (s, 1 H), 8.13 (s, 1 H) |
| 34 | 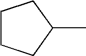 | 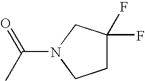 | m/z; 445 (M + H)⁺ | 1H NMR (600 MHz, CHLOROFORM-d) δ ppm 1.35-1.47 (m, 2 H), 1.49-1.60 (m, 2 H), 1.63-1.74 (m, 2 H), 1.80-1.93 (m, 4 H), 1.98-2.07 (m, 2 H), 2.34 (br. s., 2 H), 2.46-2.56 (m, 3 H), 2.81 (br. s., 2 H), 3.78-4.17 (m, 4 H), 4.34 (br. s., 1 H), 6.93-7.04 (m, 2 H), 7.53-7.61 (m, 2 H), 7.91 (br. s., 1 H), 8.25 (br. s., 1 H) |

TABLE 3-5

| | | | MS | |
|---|---|---|---|---|
| Com- | | | (ESI/APCI | |
| pound | | | Dual) | |
| No. | R1 | R | observe MH⁺ | ¹H NMR (600 MHz, CHLOROFORM-d) |
| 35 | 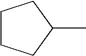 | 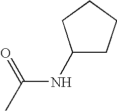 | m/z; 423 (M + H)⁺ | 1H NMR (600 MHz, CHLOROFORM-d) δ ppm 1.36-1.93 (m, 16 H), 1.97-2.14 (m, 4 H), 2.34 (br. s., 2 H), 2.47-2.55 (m, 1 H), 2.80 (br. s., 2H), 4.30-4.42 (m, 2 H), 5.70 (d, J = 7.3 Hz, 1 H), 6.95-6.99 (m, 2 H), 7.48-7.61 (m, 2 H), 7.84 (s, 1 H), 8.24 (s, 1 H) |
| 36 | 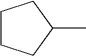 | 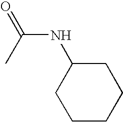 | m/z; 437 (M + H)⁺ | 1H NMR (600 MHz, CHLOROFORM-d) δ ppm 1.14-1.27 (m, 3 H), 1.36-1.91 (m, 15 H), 1.97-2.06 (m, 4 H), 2.34 (br. s., 2 H), 2.52 (t, J = 7.3 Hz, 1 H), 2.80 (br. s., 2 H), 3.89-4.00 (m, 1 H), 4.29-4.38 (m, 1 H), 5.58-5.65 (m, 1 H), 6.93-7.00 (m, 2 H), 7.52-7.58 (m, 2 H), 7.85 (s, 1 H), 8.24 (s, 1 H) |
| 37 | 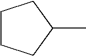 | 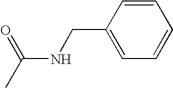 | m/z; 445 (M + H)⁺ | 1H NMR (600 MHz, CHLOROFORM-d) δ ppm 1.34-1.47 (m, 2 H), 1.48-1.61 (m, 2 H), 1.64-1.74 (m, 2 H), 1.79-1.91 (m, 4 H), 1.97-2.06 (m, 2 H), 2.27-2.40 (m, 2 H), 2.45-2.57 (m, 1 H), 2.74-2.85 (m, 2 H), 4.30-4.37 (m, 1 H), 4.58-4.65 (m, 2 H), 6.03-6.10 (m, 1 H), 6.94-7.01 (m, 2 H), 7.27-7.39 (m, 5 H), 7.51-7.59 (m, 2 H), 7.87 (s, 1 H), 8.28 (s, 1 H) |
| 38 | 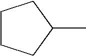 | 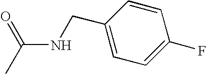 | m/z; 463 (M + H)⁺ | 1H NMR (600 MHz, CHLOROFORM-d) δ ppm 1.36-1.45 (m, 2 H), 1.49-1.60 (m, 2 H), 1.84-1.73 (m, 2 H), 1.80-1.92 (m, 4 H), 1.97-2.06 (m, 2 H), 2.35 (br. s., 2 H), 2.45-2.59 (m, 1 H), 2.80 (br. s., 2 H), 4.34 (br. s., 1 H), 4.59 (d, J = 6.0 Hz, 2 H), 6.07 (t, J = 5.7 Hz 1 H), 6.95-7.06 (m, 4 H), 7.29-7.36 (m, 2 H), 7.48-7.64 (m, 2 H), 7.87 (s, 1 H), 8.28 (s, 1 H) |
| 39 | 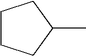 | 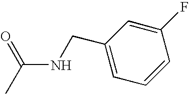 | m/z; 463 (M + H)⁺ | 1H NMR (600 MHz, CHLOROFORM-d) δ ppm 1.36-1.46 (m, 2 H), 1.50-1.60 (m, 2 H), 1.85-1.73 (m, 2 H), 1.81-1.91 (m, 4 H), 1.98-2.05 (m, 2 H), 2.34 (br. s., 2 H), 2.48-2.56 (m, 1 H), 2.80 (br. s., 2 H), 4.31-4.37 (m, 1 H), 4.62 (d, J = 6.0 Hz, 2 H), 6.07-6.13 (m, 1 H), 6.96-7.01 (m, 3 H), 7.04-7.08 (m, 1 H), 7.11-7.14 (m, 1 H), 7.28-7.35 (m, 1 H), 7.54-7.58 (m, 2 H), 7.89 (s, 1 H), 8.29 (s, 1 H) |

TABLE 3-6

| | | | MS | |
|---|---|---|---|---|
| Com- | | | (ESI/APCI | |
| pound | | | Dual) | |
| No. | R1 | R | observe MH⁺ | ¹H NMR (600 MHz, CHLOROFORM-d) |
| 40 | 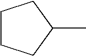 | 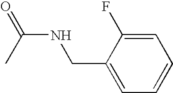 | m/z; 463 (M + H)⁺ | 1H NMR (600 MHz, CHLOROFORM-d) δ ppm 1.37-1.46 (m, 2 H), 1.50-1.61 (m, 2 H), 1.65-1.73 (m, 2 H), 1.80-1.93 (m, 4 H), 1.97-2.06 (m, 2 H), 2.34 (br. s., 2 H), 2.48-2.57 (m, 1 H), 2.80 (br. s., 2 H), 4.33 (br. s., 1 H), 4.67 (d, J = 6.0 Hz, 2 H), 6.11-6.18 (m, 1 H), 6.95-6.99 (m, 2 H), 7.04-7.09 (m, 1 H), 7.10-7.15 (m, 1 H), 7.24-7.30 (m, 1 H), 7.40-7.45 (m, 1 H), 7.52-7.57 (m, 2 H), 7.88 (s, 1 H), 8.26 (s, 1 H) |
| 41 | 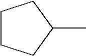 | 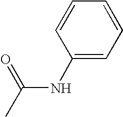 | m/z; 431 (M + H)⁺ | 1H NMR (600 MHz, CHLOROFORM-d) δ ppm 1.37-1.47 (m, 2 H), 1.51-1.61 (m, 2 H), 1.65-1.73 (m, 2 H), 1.81-1.92 (m, 4 H), 1.99-2.06 (m, 2 H), 2.34 (br. s., 2 H), 2.49-2.56 (m, 1 H), 2.81 (br. s 2 H), 4.31-4.38 (m, 1 H), 6.97-7.01 (m, 2 H), 7.14 (t, J = 7.3 Hz, 1 H), 7.36 (t, J = 8.0 Hz, 2 H), 7.49 (s, 1 H), 7.55-7.63 (m, 4 H), 8.00 (s, 1 H), 8.35 (s, 1 H) |
| 42 | 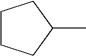 | 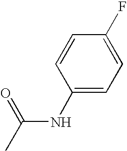 | m/z; 449 (M + H)⁺ | 1H NMR (600 MHz, CHLOROFORM-d) δ ppm 1.36-1.47 (m, 2 H), 1.50-1.62 (m, 2 H), 1.64-1.74 (m, 2 H), 1.80-1.92 (m, 4 H), 1.99-2.06 (m, 2 H), 2.35 (br. s., 2 H), 2.48-2.56 (m, 1 H), 2.81 (br. s., 2 H), 4.30-4.40 (m, 1 H), 6.97-7.02 (m, 2 H), 7.03-7.09 (m, 2 H), 7.45 (s, 1 H), 7.52-7.63 (m, 4 H), 7.99 (s, 1 H), 8.35 (s, 1 H) |
| 43 | 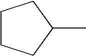 | 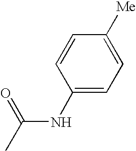 | m/z; 445 (M + H)⁺ | 1H NMR (600 MHz, CHLOROFORM-d) δ ppm 1.37-1.47 (m, 2 H), 1.50-1.61 (m, 2 H), 1.64-1.74 (m, 2 H), 1.81-1.92 (m, 4 H), 1.98-2.06 (m, 2 H), 2.29-2.40 (m, 5 H), 2.48-2.56 (m, 1 H), 2.81 (br. s., 2 H), 4.35 (br. s., 1 H), 6.96-7.01 (m, 2 H), 7.16 (d, J = 8.3 Hz, 2 H), 7.40-7.53 (m, 3 H), 7.58 (d, J = 9.2 Hz, 2 H), 7.98 (br. s., 1 H), 8.34 (s, 1 H) |
| 44 | 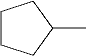 | 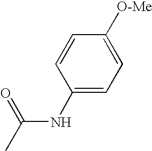 | m/z; 461 (M + H)⁺ | 1H NMR (600 MHz, CHLOROFORM-d) δ ppm 1.37-1.47 (m, 2 H), 1.50-1.61 (m, 2 H), 1.64-1.74 (m, 2 H), 1.81-1.91 (m, 4 H), 1.98-2.06 (m, 2 H), 2.34 (br. s., 2 H), 2.48-2.57 (m, 1 H), 2.81 (br. s., 2 H), 3.81 (s, 3 H), 4.31-4.38 (m, 1 H), 6.87-6.93 (m, 2 H), 6.96-7.02 (m, 2 H), 7.41 (br. s., 1 H), 7.46-7.60 (m, 4 H), 7.98 (br. s., 1 H), 8.34 (br. s., 1 H) |

TABLE 3-7

| | | | MS | |
|---|---|---|---|---|
| | | | (ESI/APCI | |
| Com- | | | Dual) | |
| pound | | | observe | |
| No. | R1 | R | MH⁺ | ¹H NMR (600 MHz, CHLOROFORM-d) |
| 45 | 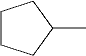 | 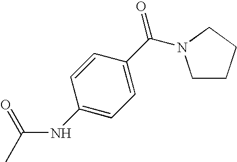 | m/z; 528 (M + H)⁺ | 1H NMR (600 MHz, CHLOROFORM-d) δ ppm 1.36-1.47 (m, 2 H), 1.51-1.62 (m, 2 H), 1.64-1.74 (m, 2 H), 1.81-1.99 (m, 8 H), 1.99-2.06 (m, 2 H), 2.34 (br. s., 2 H), 2.48-2.56 (m, 1 H), 2.81 (br. s., 2 H), 3.45 (t, J = 6.4 Hz, 2 H), 3.64 (t, J = 7.1 Hz, 2 H), 4.31 -4.39 (m, 1 H), 6.95-7.01 (m, 2 H), 7.39-7.45 (m, 2 H), 7.49-7.54 (m, 2 H), 7.57-7.61 (m, 2 H), 8.15 (s, 1 H), 8.28 (s, 1 H), 8.50 (s, 1 H) |
| 46 | 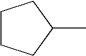 | 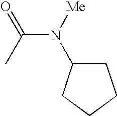 | m/z; 437 (M + H)⁺ | 1H NMR (600 MHz, CHLOROFORM-d) δ ppm 1.36-1.46 (m, 2 H), 1.48-1.78 (m, 11 H), 1.80-1.94 (m, 6 H), 1.97-2.07 (m, 2 H), 2.33 (br. s., 2 H), 2.48-2.56 (m, 1 H), 2.80 (br. s., 2 H), 3.00 (br. s., 3 H), 4.30-4.37 (m, 1 H), 6.94-7.00 (m, 2 H), 7.53-7.58 (m, 2 H), 7.81 (br. s., 1 H), 8.14 (br. s., 1 H) |
| 47 | 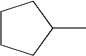 | 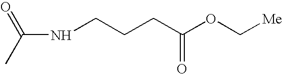 | m/z; 469 (M + H)⁺ | 1H NMR (600 MHz, CHLOROFORM-d) δ ppm 1.24 (t, J = 7.2 Hz, 3 H), 1.37-1.46 (m, 2 H), 1.51-1.60 (m, 2 H), 1.65-1.73 (m, 2 H), 1.81-1.91 (m, 4 H), 1.82-2.06 (m, 4 H), 2.34 (br. s., 2 H), 2.42-2.47 (m, 2 H), 2.48-2.56 (m, 1 H), 2.80 (br. s., 2 H), 3.46-3.51 (m, 2 H), 4.13 (d, J = 7.2 Hz, 2 H), 4.34 (br. s., 1 H), 6.33-6.38 (m, 1 H), 6.96-7.00 (m, 2 H), 7.54-7.58 (m, 2 H), 7.90 (s, 1 H), 8.25 (s, 1 H) |
| 48 | 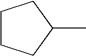 | 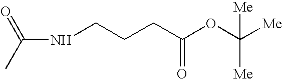 | m/z; 497 (M + H)⁺ | 1H NMR (600 MHz, CHLOROFORM-d) δ ppm 1.36-1.48 (m, 11 H), 1.50-1.59 (m, 3 H), 1.63-1.75 (m, 2 H), 1.81-1.95 (m, 6 H), 1.98-2.06 (m, 2 H), 2.29-2.41 (m, 4 H), 2.47-2.57 (m, 1 H), 2.80 (br. s., 2 H), 3.42-3.49 (m, 2 H), 4.27-4.39 (m, 1 H), 6.95-7.01 (m, 2 H), 7.52-7.59 (m, 2 H), 7.91 (s, 1 H), 8.26 (s, 1 H) |

### Example 7

### Preparation of 4-{[(1-{4-[(1-cyclopentylpiperidin-4-yl)oxy]phenyl}-1H-pyrazol-4-yl)carbonyl]amino}butanoic acid hydrochloride (Compound No. 49)

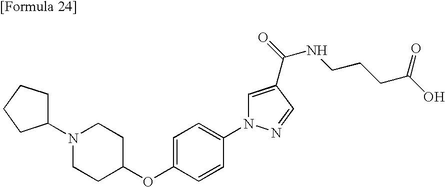

To a solution of tert-butyl 4-{[(1-{4-[(1-cyclopentylpiperidin-4-yl)oxy]phenyl}-1H-pyrazol-4-yl)carbonyl]amino}butanoate (Compound No. 48) (0.26 g) in chloroform, 4M hydrochloric acid in ethyl acetate (1.33 mL) was added under ice cooling and stirred overnight at room temperature. The reaction mixture was concentrated under reduced pressure to give the titled compound (0.88 g).

¹H NMR (600 MHz, DMSO-d₆) δ ppm 1.44-1.60 (m, 2H), 1.63-1.81 (m, 6H), 1.85-2.07 (m, 4H), 2.09-2.29 (m, 4H), 2.93-3.15 (m, 2H), 3.17-3.26 (m, 2H), 3.29-3.57 (m, 4H), 4.54-4.82 (m, 1H), 7.05-7.19 (m, 2H), 7.66-7.79 (m, 2H), 8.06 (s, 1H), 8.19 (t, J=5.5 Hz, 1H), 8.79 (s, 1H), 10.51 (br. s, 1H)

MS (ESI/APCI Dual) (Positive) m/z; 441 (M+H)⁺

### Example 8

### Preparation of 1-tert-butyl-4-{4-[4-(pyrrolidin-1-ylcarbonyl)-1H-pyrazol-1-yl]phenoxy}piperidine (Compound No. 50)

### (1) Preparation of 1-tert-butyl-4-(4-iodophenoxy)piperidine

The same procedure as shown in Example 1-(1) was repeated to give the titled compound, except that tert-butyl 4-hydroxypiperidine-1-carboxylate was replaced with 1-tert-butyl-4-hydroxypiperidine (which may be synthesized according to the method described in Journal of Organic Chemistry, 2005, vol. 70, pages 1930-1933).

### (2) Preparation of 1-tert-butyl-4-{4-[4-(pyrrolidin-1-ylcarbonyl)-1H-pyrazol-1-yl]phenoxy}piperidine (Compound No. 50)

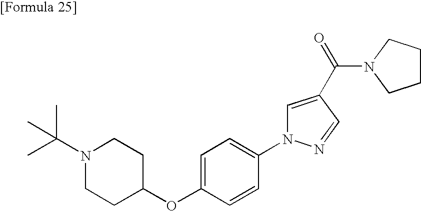

The same procedure as shown in Example 1-(2) was repeated to give the titled compound, except that tert-butyl 4-(4-iodophenoxy)piperidine-1-carboxylate was replaced with 1-tert-butyl-4-(4-iodophenoxy)piperidine obtained in Example 8-(1).

¹H NMR (600 MHz, DMSO-d₆) δ ppm 1.11 (s, 1H), 1.73-2.12 (m, 8H), 2.46 (br.s, 2H), 2.88 (br.s, 2H), 3.65 (t, J=6.9 Hz, 2H), 3.73 (t, J=6.7 Hz, 2H), 4.32 (br.s, 1H), 6.97 (d, J=9.7 Hz, 2H), 7.56 (d, J=9.2 Hz, 2H), 7.94 (s, 1H), 8.26 (s, 1H)

MS (ESI/APCI Dual) (Positive) m/z; 397 (M+H)⁺

### Example 9

### Preparation of 1-{4-[(1-cyclopropylpiperidin-4-yl)oxy]phenyl}-N-(4-fluorophenyl)-1H-pyrazole-4-carboxamide (Compound No. 51)

### (1) Preparation of 1-cyclopropyl-4-(4-iodophenoxy)piperidine

The same procedure as shown in Example 1-(1) was repeated to give the titled compound, except that tert-butyl 4-hydroxypiperidine-1-carboxylate was replaced with 1-cyclopropyl-4-hydroxypiperidine (which may be synthesized according to the method described in WO2005117865).

### (2) Preparation of 1-{4-[(1-cyclopropylpiperidin-4-yl)oxy]phenyl}-N-(4-fluorophenyl)-1H-pyrazole-4-carboxamide (Compound No. 51)

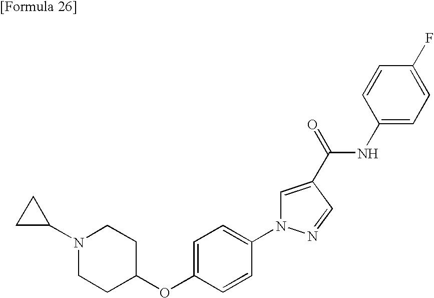

The same procedure as shown in Example 1-(3) was repeated to give the titled compound, except that tert-butyl 4-(4-iodophenoxy)piperidine-1-carboxylate was replaced with 1-cyclopropyl-4-(4-iodophenoxy)piperidine obtained in Example 9-(1), and 4-(pyrrolidin-1-ylcarbonyl)-1H-pyrazole was replaced with 1H-pyrazole-4-carboxylic acid (4-fluorophenyl)amide.

¹H NMR (600 MHz, CHLOROFORM-d) δ ppm 0.40-0.49 (m, 4H), 1.61-1.66 (m, 1H), 1.77-1.84 (m, 2H), 1.95-2.02 (m, 2H), 2.47-2.54 (m, 2H), 2.87-2.95 (m, 2H), 4.34-4.40 (m, 1H), 7.01 (d, J=8.7 Hz, 2H), 7.07 (t, J=8.7 Hz, 2H), 7.44 (s, 1H), 7.55-7.62 (m, 4H), 7.99 (s, 1H), 8.36 (s, 1H)

MS (ESI/APCI Dual) (Positive) m/z; 421 (M+H)⁺

### Example 10

### Preparation of N-(4-fluorophenyl)-1-(4-{[1-(1-methylethyl)piperidin-4-yl]oxy}phenyl)-1H-pyrazole-4-carboxamide (Compound No. 52)

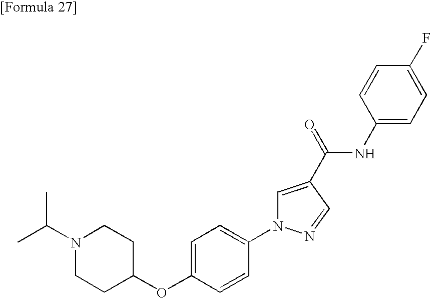

The same procedure as shown in Example 1-(3) was repeated to give the titled compound, except that tert-butyl 4-(4-iodophenoxy)piperidine-1-carboxylate was replaced with 1-isopropyl-4-(4-iodophenoxy)piperidine (which may be synthesized in the same manner as shown in Example 1-(1)), and 4-(pyrrolidin-1-ylcarbonyl)-1H-pyrazole was replaced with 1H-pyrazole-4-carboxylic acid (4-fluorophenyl)amide.

¹H NMR (600 MHz, CHLOROFORM-d) δ ppm 1.07 (d, J=6.4 Hz, 6H), 1.80-1.88 (m, 2H), 2.00-2.07 (m, 2H), 2.37-2.46 (m, 2H), 2.72-2.83 (m, 3H), 4.31-4.37 (m, 1H), 7.01 (d, J=8.7 Hz, 2H), 7.07 (t, J=8.7 Hz, 2H), 7.44 (s, 1H), 7.52-7.65 (m, 4H), 7.99 (s, 1H), 8.36 (s, 1H)

MS (ESI/APCI Dual) (Positive) m/z; 423 (M+H)⁺

### Example 11

### Preparation of 1-{4-[(1-cyclopentylpyrrolidin-3-yl)oxy]phenyl}-4-(pyrrolidin-1-ylcarbonyl)-1H-pyrazole (Compound No. 53)

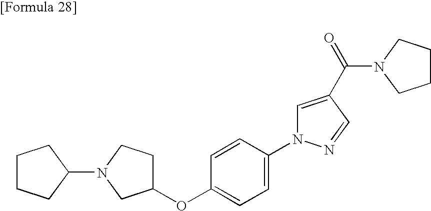

The same procedure as shown in Example 1 was repeated to give the titled compound, except that tert-butyl 4-hydroxypiperidine-1-carboxylate was replaced with tert-butyl 4-hydroxypyrrolidine-1-carboxylate.

¹H NMR (600 MHz, CHLOROFORM-d) δ ppm 1.39-1.61 (m, 4H), 1.64-1.76 (m, 2H), 1.78-1.86 (m, 2H), 1.89-2.06 (m, 5H), 2.28-2.38 (m, 1H), 2.43-2.60 (m, 2H), 2.74-2.87 (m, 2H), 2.89-2.98 (m, 1H), 3.65 (t, J=6.9 Hz, 2H), 3.73 (t, J=6.9 Hz, 2H), 4.80-4.86 (m, 1H), 6.87-6.95 (m, 2H), 7.52-7.60 (m, 2H), 7.94 (s, 1H), 8.25 (s, 1H)

MS (ESI/APCI Dual) (Positive) m/z; 395 (M+H)⁺

### Example 12

### Preparation of 1-{4-[(1-cyclobutylpyrrolidin-3-yl)oxy]phenyl}-4-(pyrrolidin-1-ylcarbonyl)-1H-pyrazole (Compound No. 54)

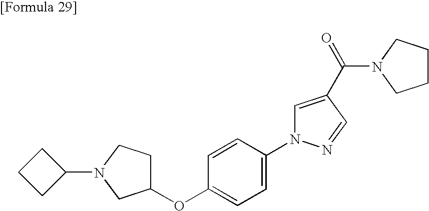

The same procedure as shown in Example 1 was repeated to give the titled compound, except that tert-butyl 4-hydroxypiperidine-1-carboxylate was replaced with tert-butyl 4-hydroxypyrrolidine-1-carboxylate, and cyclopentanone was replaced with cyclobutanone.

¹H NMR (600 MHz, CHLOROFORM-d) δ ppm 1.65-1.79 (m, 2H), 1.89-2.06 (m, 9H), 2.27-2.36 (m, 1H), 2.42-2.50 (m, 1H), 2.70-2.76 (m, 2H), 2.81-2.88 (m, 1H), 2.92-2.99 (m, 1H), 3.65 (t, J=6.9 Hz, 2H), 3.73 (t, J=6.6 Hz, 2H), 4.81-4.86 (m, 1H), 6.89-6.94 (m, 2H), 7.53-7.58 (m, 2H), 7.94 (s, 1H), 8.26 (s, 1H)

MS (ESI/APCI Dual) (Positive) m/z; 381 (M+H)⁺

### Example 13

### Preparation of 1-cyclopentyl-4-{3-fluoro-4-[4-(pyrrolidin-1-ylcarbonyl)-1H-pyrazol-1-yl]phenoxy}piperidine (Compound No. 55)

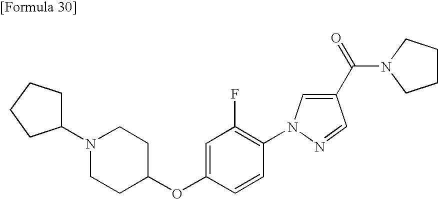

The same procedure as shown in Example 1 was repeated to give the titled compound, except that 4-iodophenol was replaced with 3-fluoro-4-iodophenol.

MS (ESI/APCI Dual) (Positive) m/z; 427 (M+H)⁺

### Example 14

### Preparation of 1-cyclobutyl-4-{3-fluoro-4-[4-(pyrrolidin-1-ylcarbonyl)-1H-pyrazol-1-yl]phenoxy}piperidine (Compound No. 56)

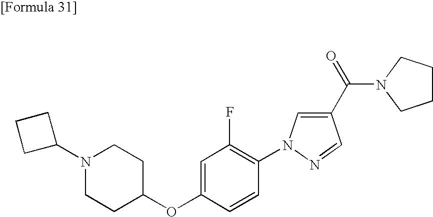

The same procedure as shown in Example 1 was repeated to give the titled compound, except that 4-iodophenol was replaced with 3-fluoro-4-iodophenol, and cyclopentanone was replaced with cyclobutanone.

MS (ESI/APCI Dual) (Positive) m/z; 413 (M+H)⁺

### Example 15

### Preparation of 1-cyclopentyl-4-{2-fluoro-4-[4-(pyrrolidin-1-ylcarbonyl)-1H-pyrazol-1-yl]phenoxy}piperidine (Compound No. 57)

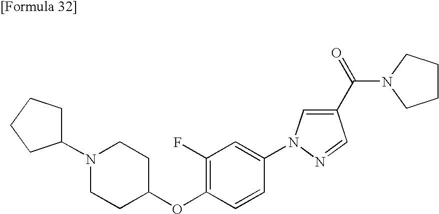

The same procedure as shown in Example 1 was repeated to give the titled compound, except that 4-iodophenol was replaced with 2-fluoro-4-iodophenol.

¹H NMR (600 MHz, CHLOROFORM-d) δ ppm 1.37-1.46 (m, 2H), 1.49-1.62 (m, 2H), 1.64-1.75 (m, 2H), 1.82-2.09 (m, 10H), 2.34 (br. s, 2H), 2.48-2.56 (m, 1H), 2.81 (br. s, 2H), 3.65 (t, J=6.9 Hz, 2H), 3.73 (t, J=6.6 Hz, 2H), 4.33 (br. s, 1H), 7.06 (t, J=8.7 Hz, 1H), 7.33-7.38 (m, 1H), 7.48 (dd, J=11.7, 2.5 Hz, 1H), 7.95 (s, 1H), 8.27 (s, 1H)

MS (ESI/APCI Dual) (Positive) m/z; 427 (M+H)⁺

### Example 16

### Preparation of 1-cyclobutyl-4-{2-fluoro-4-[4-(pyrrolidin-1-ylcarbonyl)-1H-pyrazol-1-yl]phenoxy}piperidine (Compound No. 58)

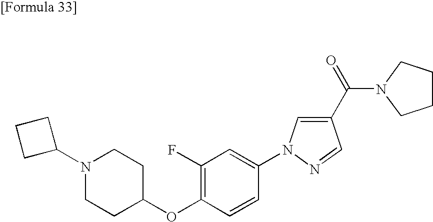

The same procedure as shown in Example 1 was repeated to give the titled compound, except that 4-iodophenol was replaced with 2-fluoro-4-iodophenol, and cyclopentanone was replaced with cyclobutanone.

¹H NMR (600 MHz, CHLOROFORM-d) δ ppm 1.62-1.76 (m, 2H), 1.81-2.09 (m, 12H), 2.16 (br. s, 2H), 2.62 (br. s, 2H), 2.70-2.78 (m, 1H), 3.65 (t, J=6.9 Hz, 2H), 3.73 (t, J=6.9 Hz, 2H), 4.33 (br. s, 1H), 7.06 (t, J=8.7 Hz, 1H), 7.33-7.36 (m, 1H), 7.48 (dd, J=11.9, 2.8 Hz, 1H), 7.95 (s, 1H), 8.27 (s, 1H)

MS (ESI/APCI Dual) (Positive) m/z; 413 (M+H)⁺

### Example 17

### Preparation of 1-cyclopentyl-4-{3-methyl-4-[4-(pyrrolidin-1-ylcarbonyl)-1H-pyrazol-1-yl]phenoxy}piperidine (Compound No. 59)

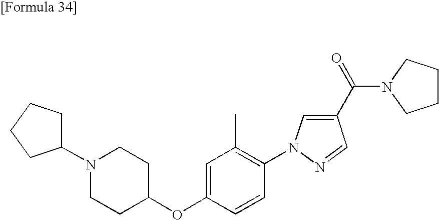

The same procedure as shown in Example 1 was repeated to give the titled compound, except that 4-iodophenol was replaced with 4-iodo-3-methylphenol.

¹H NMR (600 MHz, CHLOROFORM-d) δ ppm 1.35-1.47 (m, 2H), 1.49-1.60 (m, 2H), 1.62-1.74 (m, 2H), 1.79-1.97 (m, 6H), 1.97-2.06 (m, 4H), 2.15 (s, 3H), 2.35 (br. s, 2H), 2.47-2.57 (m, 1H), 2.79 (br. s, 2H), 3.65 (t, J=6.9 Hz, 2H), 3.73 (t, J=6.6 Hz, 2H), 4.34 (br. s, 1H), 6.75-6.85 (m, 2H), 7.20 (d, J=8.3 Hz, 1H), 7.97 (s, 2H)

MS (ESI/APCI Dual) (Positive) m/z; 423 (M+H)⁺

### Example 18

### Preparation of 1-cyclobutyl-4-{3-methyl-4-[4-(pyrrolidin-1-ylcarbonyl)-1H-pyrazol-1-yl]phenoxy}piperidine (Compound No. 60)

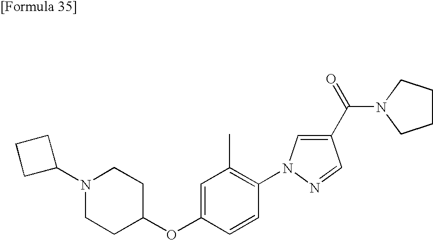

The same procedure as shown in Example 17 was repeated to give the titled compound, except that 4-iodophenol was replaced with 4-iodo-3-methylphenol, and cyclopentanone was replaced with cyclobutanone.

¹H NMR (600 MHz, CHLOROFORM-d) δ ppm 1.62-1.76 (m, 2H), 1.78-2.09 (m, 12H), 2.11-2.24 (m, 5H), 2.60 (br. s, 2H), 2.69-2.78 (m, 1H), 3.65 (t, J=7.1 Hz, 2H), 3.73 (t, J=6.9 Hz, 2H), 4.35 (br. s, 1H), 6.76-6.80 (m, 1H), 6.81-6.84 (m, 1H), 7.16-7.23 (m, 1H), 7.97 (s, 2H)

MS (ESI/APCI Dual) (Positive) m/z; 409 (M+H)⁺

### Example 19

### Preparation of 1-{4-[(1-cyclobutylpiperidin-4-yl)oxy]phenyl}-1H-pyrazole-4-carboxamide (Compound No. 61)

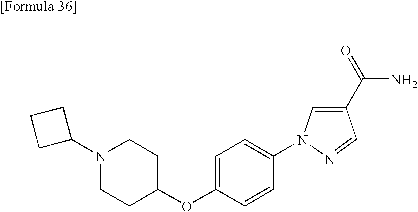

To a solution of 1-{4-[(1-cyclobutylpiperidin-4-yl)oxy]phenyl}-1H-pyrazole-4-carboxylic acid obtained in Example 5-(1) (1.00 g), 1-hydroxybenzotriazole monohydrate (0.535 g) and 1-ethyl-3-(3-dimethylaminopropyl)carbodiimide hydrochloride (0.673 g) in N,N-dimethylformamide, aqueous ammonia (25%, 0.5 ml) was added at room temperature and stirred overnight at room temperature. After addition of saturated aqueous sodium bicarbonate, the reaction mixture was stirred for 1 hour and filtered to collect the crystal. The resulting crystal was washed with water and diisopropyl ether, and then dried to give the titled compound (0.988 g) as a colorless powder.

### Example 20

### Preparation of 1-{4-[(1-cyclopentylpiperidin-4-yl)oxy]phenyl}-1H-pyrazole-4-carbonitrile (Compound No. 62)

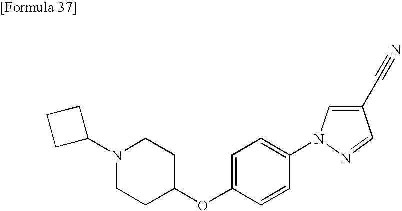

To a solution of 1-{4-[(1-cyclobutylpiperidin-4-yl)oxy]phenyl}-1H-pyrazole-4-carboxamide obtained in Example 19 (0.154 g) in N,N-dimethylformamide, thionyl chloride (0.134 g) was added under ice cooling, followed stirring on ice for 30 minutes and then at room temperature for an additional 4 hours. To the reaction mixture, thionyl chloride (0.825 g) was added under ice cooling and stirred at room temperature for 2 hours. The reaction mixture was cooled on ice, diluted with water and saturated aqueous sodium bicarbonate, adjusted to pH 8 and then extracted with ethyl acetate. The organic layer was washed with saturated aqueous sodium chloride, dried over sodium sulfate and concentrated under reduced pressure. The resulting residue was purified by silica gel column chromatography (eluting solvent: n-hexane:ethyl acetate=1:1) to give the titled compound (0.106 g) as a colorless solid.

¹H NMR (600 MHz, CHLOROFORM-d) δ ppm 1.63-1.75 (m, 2H), 1.79-1.92 (m, 4H), 1.96-2.07 (m, 4H), 2.16 (br. s, 2H), 2.62 (br. s, 2H), 2.70-2.77 (m, J=7.9, 7.9, 7.8, 7.6 Hz, 1H), 4.35 (br. s, 1H), 6.96-7.01 (m, 2H), 7.51-7.55 (m, 2H), 7.95 (s, 1H), 8.18 (s, 1H)

MS (ESI/APCI Dual) (Positive) m/z; 323 (M+H)⁺

### Example 21

### Preparation of 1-cyclobutyl-4-[4-(4-nitro-1H-pyrazol-1-yl)phenoxy]piperidine (Compound No. 63)

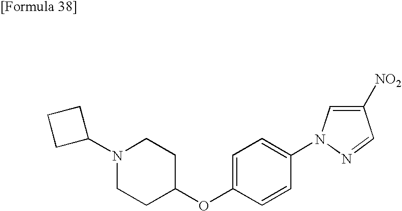

A mixture of 1-cyclobutyl-4-(4-iodophenoxy)piperidine (5.68 g; which may be synthesized in the same manner as shown in Example 1-(1), except that tert-butyl 4-hydroxypiperidine-1-carboxylate was replaced with 1-cyclobutyl-4-hydroxypiperidine), 4-nitropyrazole (1.98 g), rac-trans-N,N′-dimethylcyclohexane-1,2-diamine (0.905 g), copper iodide (0.303 g) and cesium carbonate (10.9 g) in N,N-dimethylformamide (10 ml) was stirred at 130° C. for 1.5 hours. The reaction mixture was cooled to room temperature, diluted with water and extracted with ethyl acetate. The organic layer was washed with saturated aqueous sodium chloride, dried over sodium sulfate and concentrated under reduced pressure. The resulting residue was purified by silica gel column chromatography (eluting solvent: n-hexane:ethyl acetate=2:1), and the resulting crystal was washed with diisopropyl ether to give the titled compound (2.34 g) as a colorless solid.

¹H NMR (600 MHz, CHLOROFORM-d) δ ppm 1.63-1.75 (m, 2H), 1.79-1.93 (m, 4H), 1.97-2.08 (m, 4H), 2.17 (br. s, 2H), 2.62 (br. s, 2H), 2.70-2.78 (m, 1H), 4.36 (br. s, 1H), 6.97-7.03 (m, 2H), 7.54-7.60 (m, 2 μl), 8.22 (s, 1H), 8.50 (s, 1H)

MS (ESI/APCI Dual) (Positive) m/z; 343 (M+H)⁺

### Example 22

### Preparation of 4-chloro-N-(1-{4-[(1-cyclobutylpiperidin-4-yl)oxy]phenyl}-1H-pyrazol-4-yl)butaneamide (Compound No. 64)

### (1) Preparation of 1-{4-[(1-cyclopentylpiperidin-4-yl)oxy]phenyl}-1H-pyrazole-4-amine

To a solution of 1-cyclobutyl-4-[4-(4-nitro-1H-pyrazol-1-yl)phenoxy]piperidine obtained in Example 21 (1.00 g) in methanol (20 ml), palladium/carbon (5%, 0.10 g) was added and stirred overnight at room temperature under a hydrogen atmosphere. After filtration through celite, the filtrate was concentrated under reduced pressure and the resulting residue was purified by silica gel column chromatography (eluting solvent: ethyl acetate) to give the titled compound (0.883 g) as a light-orange solid.

### (2) Preparation of 4-chloro-N-(1-{4-[(1-cyclobutylpiperidin-4-yl)oxy]phenyl}-1H-pyrazol-4-yl)butaneamide (Compound No. 64)

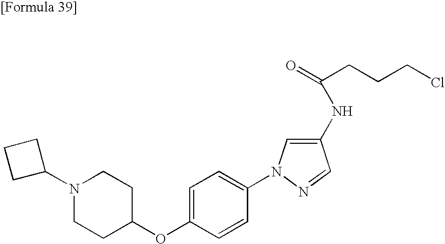

To a solution of 1-{4-[(1-cyclobutylpiperidin-4-yl)oxy]phenyl}-1H-pyrazole-4-amine obtained in Example 22-(1) (0.300 g) in chloroform (3.0 ml), pyridine (0.152 g) and 4-chlorobutyric acid chloride (0.149 g) were added at room temperature and stirred at room temperature for 30 minutes. The reaction mixture was diluted with saturated aqueous ammonium chloride, extracted with chloroform, washed with water and saturated aqueous sodium chloride, dried over sodium sulfate and then concentrated under reduced pressure. The resulting residue was purified by silica gel column chromatography (eluting solvent: n-hexane:ethyl acetate=1:1) to give the titled compound (0.366 g) as a light-yellow amorphous substance.

¹H NMR (600 MHz, CHLOROFORM-d) δ ppm 1.63-1.76 (m, 2H), 1.78-1.95 (m, 4H), 1.95-2.09 (m, 4H), 2.09-2.24 (m, 4H), 2.53-2.69 (m, 4H), 2.69-2.80 (m, 1H), 3.61-3.68 (m, 2H), 4.32 (br. s, 1H), 6.92-7.00 (m, 2H), 7.28 (s, 1H), 7.51-7.58 (m, 3H), 8.37 (s, 1H)

MS (ESI/APCI Dual) (Positive) m/z; 417 (M+H)⁺

### Example 23

### Preparation of 1-(1-{4-[(1-cyclopentylpiperidin-4-yl)oxy]phenyl}-1H-pyrazol-4-yl)pyrrolidin-2-one (Compound No. 65)

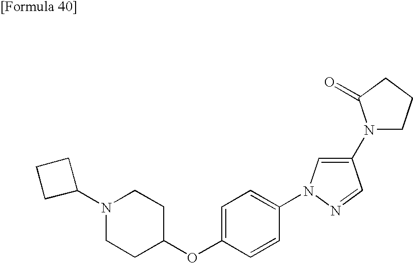

To a solution of 4-chloro-N-(1-{4-[(1-cyclobutylpiperidin-4-yl)oxy]phenyl}-1H-pyrazol-4-yl)butaneamide obtained in Example 22-(2) (0.366 g) in tetrahydrofuran (3.0 ml), sodium hydride (0.176 g) was added at room temperature and heated under reflux for 30 minutes in an oil bath (oil temperature: 90° C.). The reaction mixture was cooled on ice, diluted with saturated aqueous sodium bicarbonate, extracted with chloroform, washed with water and saturated aqueous sodium chloride, dried over sodium sulfate and then concentrated under reduced pressure. The resulting residue was purified by silica gel column chromatography (eluting solvent: chloroform:methanol:aqueous ammonia=10:1:0.1), and the resulting crystal was washed with ethyl acetate to give the titled compound (0.082 g) as a light-yellow solid.

¹H NMR (600 MHz, CHLOROFORM-d) δ ppm 1.63-1.75 (m, 2H), 1.78-1.93 (m, 4H), 1.96-2.08 (m, 4H), 2.11-2.27 (m, 4H), 2.54-2.66 (m, 4H), 2.69-2.77 (m, 1H), 3.80 (t, J=7.1 Hz, 2H), 4.33 (br. s, 1H), 6.93-6.98 (m, 2H), 7.54-7.59 (m, 2H), 7.64 (s, 1H), 8.43 (s, 1H)

MS (ESI/APCI Dual) (Positive) m/z; 381 (M+H)⁺

### Example 24

### Preparation of 2-chloroethyl {1-[4-(1-cyclobutylpiperidin-4-yloxy)phenyl]-1H-pyrazol-4-yl}-carbamic acid (Compound No. 66)

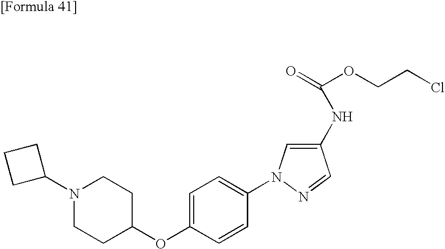

The same procedure as shown in Example 22 was repeated to give the titled compound, except that 4-chlorobutyric acid chloride was replaced with 2-chloroethyl chloroformate.

¹H NMR (200 MHz, CHLOROFORM-d) δ ppm 1.52-2.23 (m, 12H), 2.55-2.83 (m, 3H), 3.75 (t, J=6.0 Hz, 2H), 4.27-4.41 (m, 1H), 4.43 (t, J=6.0 Hz, 2H), 6.62-6.80 (m, 1H), 6.85-7.00 (m, 2H), 7.48-7.60 (m, 3H), 8.09 (s, 1H)

MS (ESI/APCI Dual) (Positive) m/z; 419 (M+H)⁺

### Example 25

### Preparation of 3-(1-{4-[(1-cyclopentylpiperidin-4-yl)oxy]phenyl}-1H-pyrazol-4-yl)-1,3-oxazolidin-2-one (Compound No. 67)

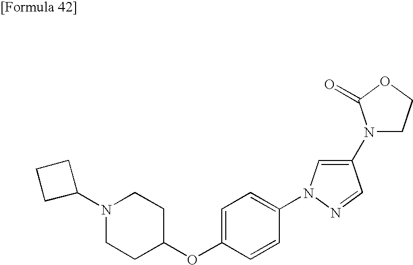

The same procedure as shown in Example 23 was repeated to give the titled compound, except that 4-chloro-N-(1-{4-[(1-cyclobutylpiperidin-4-yl)oxy]phenyl}-1H-pyrazol-4-yl)butaneamide was replaced with 2-chloroethyl {1-[4-(1-cyclobutylpiperidin-4-yloxy)phenyl]-1H-pyrazol-4-yl}-carbamic acid.

¹H NMR (600 MHz, CHLOROFORM-d) δ ppm 1.45-2.22 (m, 12H), 2.50-2.82 (m, 3H), 3.99 (t, J=8.5 Hz, 2H), 4.22-4.41 (m, 1H), 4.55 (t, J=8.5 Hz, 2H), 6.92-7.03 (m, 2H), 7.56-7.64 (m, 3H), 8.20 (s, 1H)

MS (ESI/APCI Dual) (Positive) m/z; 383 (M+H)⁺

### Example 26

### Preparation of {1-[4-(1-cyclobutylpiperidin-4-yloxy)phenyl]-1H-pyrazol-3-yl}pyrrolidin-1-yl-methanone (Compound No. 68)

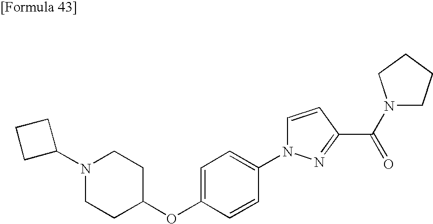

The same procedure as shown in Example 21 was repeated to give the titled compound, except that 4-nitropyrazole was replaced with 3-(pyrrolidin-1-ylcarbonyl)-1H-pyrazole.

¹H NMR (600 MHz, CHLOROFORM-d) δ ppm 1.63-1.76 (m, 4H), 1.79-2.25 (m, 16H), 2.56-2.78 (m, 3H), 3.69 (t, J=6.9 Hz, 2H), 4.04 (t, J=6.6 Hz, 2H), 4.34 (br. s., 1H), 6.96-7.00 (m, 3H), 7.58 (d, J=9.2 Hz, 2H), 7.81 (d, J=2.8 Hz, 1H)

MS (ESI/APCI Dual) (Positive) m/z; 395 (M+H)⁺

### Test Example 1

### H3 Receptor Binding Test

A membrane preparation of human H3 receptor-expressing CHO—K1 cells (Euroscreen, ES-392-M, 15 μg protein/200 μl), R(−)-α-methyl[³H]histamine (Amersham, TRK-1017, specific activity: 1.74 TBq/mmol, 2 nM) and a test drug were reacted at room temperature for 1 hour. After completion of the reaction, the reaction mixture was subjected to suction filtration through a 0.3% polyethyleneimine-treated glass filter (GF/C). The glass filter was washed five times with 50 mM Tris-HCl washing solution (pH 7.4) containing 0.1% BSA and 5 mM magnesium chloride. After washing, the glass filter was dried and a scintillator was added thereto, followed by measurement of radioactivity on the filter using a liquid scintillation counter.

Binding of R(−)-α-methyl[³H]histamine in the presence of 10 μM R(−)-α-methylhistamine was defined as non-specific binding, and the difference between total binding and non-specific binding of R(−)-α-methyl[³H]histamine was defined as specific binding of R(−)-α-methyl[³H]histamine. A fixed concentration (2 nM) of R(−)-α-methyl[³H]histamine was reacted under the above conditions with each test drug at various concentrations to obtain an inhibition curve. The inhibition curve was used to determine the concentration (IC₅₀) of each test drug required for 50% inhibition of R(−)-α-methyl[³H]histamine binding. The results obtained are shown in Table 4.

TABLE 4

| | H3 receptor binding test |
|---|---|
| Compound No. | IC₅₀ (nM) |
| 1 | 10.4 |
| 5 | 8.1 |
| 14 | 7.1 |
| 16 | 5.7 |
| 18 | 14.7 |
| 19 | 6.2 |
| 68 | 7.1 |

### Test Example 2

### [³⁵S]GTP-γ-S Binding Test

A human H3 receptor membrane preparation (7.5 μg protein/100 μl), 30 μM GDP, 100 μM R(−)-α-methylhistamine and a test compound were reacted at room temperature for 30 minutes. After completion of the reaction, [³⁵S]GTP-γ-S (0.2 nM) was added and reacted for an additional 30 minutes. After completion of the reaction, the reaction mixture was subjected to suction filtration through a glass filter (GF/C). The glass filter was washed three times with 20 mM HEPES washing solution (pH 7.4) containing 100 mM sodium chloride and 1 mM magnesium chloride. After washing, the glass filter was dried and a scintillator was added thereto, followed by measurement of radioactivity on the filter using a liquid scintillation counter.

Binding of [³⁵S]GTP-γ-S in the absence of R(−)-α-methylhistamine was defined as non-specific binding, and the difference between total binding in the presence of R(−)-α-methylhistamine and non-specific binding was defined as specific binding of [³⁵S]GTP-γ-S. Fixed concentrations of [³⁵S]GTP-γ-S (0.2 nM) and R(−)-α-methylhistamine (100 μM) were reacted under the above conditions with each test drug at various concentrations to obtain an inhibition curve. The inhibition curve was used to determine the concentration (IC₅₀) of each test drug required for 50% inhibition of [³⁵S]GTP-γ-S binding. The results obtained are shown in Table 5.

TABLE 5

| | [³⁵S]GTP-γ-S binding test |
|---|---|
| Compound No. | IC₅₀ (nM) |
| 1 | 2.6 |
| 14 | 1.6 |
| 16 | 1.1 |
| 68 | 1.2 |

### Test Example 3

### Social Recognition Test

In this test, male SD rats were used. Adult rats were placed in a Perspex test box and acclimated for 30 minutes. Then, young rats were placed in the same test box, and the time taken for the adult rats to show exploratory behavior to the young rats during 5 minutes was measured (first exploratory behavior time). The exploratory behavior measured included sniffing, grooming, and closely following. After a 5 minute exploration, the adult and young rats were removed from the test box and returned to their respective home cages. After 90 minutes, the adult rats were placed again in the same test box as used in the first exploratory behavior test and acclimated for 30 minutes. Then, the same young rats as used in the first test were placed in the test box, and the time taken for the adult rats to show exploratory behavior to the young rats during 5 minutes was measured (second exploratory behavior time). The ratio of the second exploratory behavior time to the first exploratory behavior time (second/first exploratory behavior time ratio) was taken as an indicator of social recognition. The compound of the present invention was orally administered to the adult rats immediately after their first exploratory behavior. The compound of the present invention was dissolved in 0.03 N hydrochloric acid and tested at 0.1, 0.3 and 1 mg/kg. The results indicated that Compound No. 1 significantly reduced the second/first exploratory behavior time ratio at 0.3 and 1 mg/kg when compared to the vehicle group, and showed an enhancing effect on social recognition.

### INDUSTRIAL APPLICABILITY

The present invention enables the provision of novel pyrazole derivatives which show strong binding inhibition for histamine H3 receptors and are useful for prevention and treatment of histamine H3 receptor-mediated disorders such as dementia, Alzheimer's disease, attention-deficit hyperactivity disorder, schizophrenia, epilepsy, central convulsion, eating disorders, obesity, diabetes, hyperlipidemia, sleep disorders, narcolepsy, sleep apnea syndrome, circadian rhythm disorder, depression, allergic rhinitis or other diseases.

1. A pyrazole derivative represented by formula (I) or a pharmaceutically acceptable salt thereof: 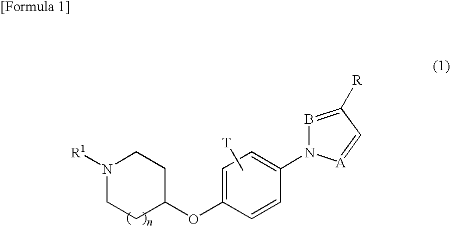
{wherein
A and B, which are different from each other, each represent a carbon atom or a nitrogen atom,
R¹ represents C₁-C₆ alkyl (wherein said C₁-C₆ alkyl may be substituted with C₃-C₈ cycloalkyl or hydroxyl); C₃-C₈ cycloalkyl (wherein said C₃-C₈ cycloalkyl may be substituted with C₁-C₆ alkyl or hydroxyl) or C₃-C₈ alkenyl,
n represents an integer of 0 to 2,
T represents a hydrogen atom; halogen or C₁-C₆ alkyl,
R represents a group represented by any of the following structural formulae (I) to (VI): 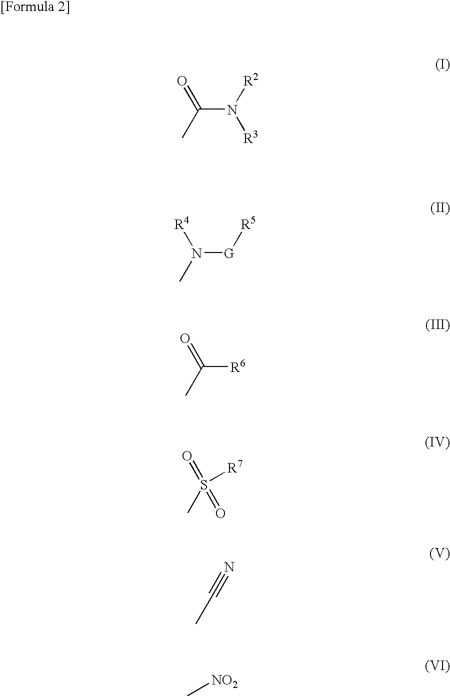
R² and R³, which may be the same or different, each represent a hydrogen atom; Cᵣ C₆ alkyl (wherein said C₁-C₆ alkyl may be substituted with halogen; C₃-C₈ cycloalkyl; hydroxyl; C₂-C₇ alkoxycarbonyl or carboxy); C₃-C₈ cycloalkyl (wherein said C₃-C₈ cycloalkyl may be substituted with halogen; C₁-C₆ alkyl or hydroxyl) or a group represented by —(CH₂)ₘ—Ar, or
R² and R³ are attached to each other together with their adjacent nitrogen atom to form a 4- to 7-membered saturated heterocyclic ring which may contain, as its ring members, one or more nitrogen, oxygen or sulfur atoms in addition to said adjacent nitrogen atom (wherein said saturated heterocyclic ring may be substituted with halogen; C₁-C₆ alkyl; C₁-C₆ alkoxy or hydroxyl),
Ar represents aryl (wherein said aryl may be substituted with halogen; C₁-C₆ alkyl; C₁-C₆ alkoxy; hydroxyl; C₂-C₇ alkoxycarbonyl; cyano; C₂-C₇ alkylaminocarbonyl; C₃-C₁₃ dialkylaminocarbonyl; carbamoyl or carbonyl which is attached via a ring nitrogen atom to a 3- to 7-membered saturated heterocyclic ring which has at least one nitrogen atom as its ring member and may contain one or more additional nitrogen, oxygen or sulfur atoms) or heteroaryl (wherein said heteroaryl may be substituted with halogen; C₁-C₆ alkyl; C₁-C₆ alkoxy; hydroxyl; C₂-C₇ alkoxycarbonyl; cyano; C₂-C₇ alkylaminocarbonyl; C₃-C₁₃ dialkylaminocarbonyl; carbamoyl or carbonyl which is attached via a ring nitrogen atom to a 3- to 7-membered saturated heterocyclic ring which has at least one nitrogen atom as its ring member and may contain one or more additional nitrogen, oxygen or sulfur atoms),
m represents an integer of 0 to 2,
G represents —CO— or —SO₂—,
R⁴ represents a hydrogen atom or C₁-C₆ alkyl,
R⁵ represents C₁-C₆ alkyl (wherein said C₁-C₆ alkyl may be substituted with halogen; C₃-C₈ cycloalkyl; C₁-C₆ alkoxy or hydroxyl); C₃-C₈ cycloalkyl (wherein said C₃-C₈ cycloalkyl may be substituted with halogen; C₁-C₆ alkyl; C₁-C₆ alkoxy or hydroxyl); aryl (wherein said aryl may be substituted with halogen; alkyl; C₁-C₆ alkoxy; hydroxyl or cyano) or heteroaryl (wherein said heteroaryl may be substituted with halogen; alkyl; C₁-C₆ alkoxy; hydroxyl or cyano), or
R⁴ and R⁵ may be attached to each other together with their adjacent nitrogen atom and carbonyl carbon to form a 5- to 7-membered saturated heterocyclic ring which may contain, as its ring members, one or more nitrogen, oxygen or sulfur atoms in addition to said adjacent nitrogen atom (wherein said saturated heterocyclic ring may be substituted with halogen; C₁-C₆ alkyl; C₁-C₆ alkoxy; hydroxyl or oxo),
R⁶ represents C₁-C₆ alkyl; C₃-C₈ cycloalkyl; C₁-C₆ alkoxy; aryl (wherein said aryl may be substituted with halogen; alkyl; C₁-C₆ alkoxy; hydroxyl or cyano) or heteroaryl (wherein said heteroaryl may be substituted with halogen; C₁-C₆ alkyl; C₁-C₆ alkoxy; hydroxyl or cyano), and
R⁷ represents C₁-C₆ alkyl; C₁-C₆ alkoxy; amino; C₁-C₆ alkylamino; C₂-C₁₂ dialkylamino; a 4- to 7-membered saturated heterocyclic ring (wherein said saturated heterocyclic ring may be substituted with halogen; alkyl; C₁-C₆ alkoxy; hydroxyl or cyano); aryl (wherein said aryl may be substituted with halogen; alkyl; C₁-C₆ alkoxy; hydroxyl or cyano) or heteroaryl (wherein said heteroaryl may be substituted with halogen; C₁-C₆ alkyl; C₁-C₆ alkoxy; hydroxyl or cyano)}.

2. The pyrazole derivative or pharmaceutically acceptable salt thereof according to claim 1, which is represented by formula (I) wherein R is represented by structural formula (I).

3. The pyrazole derivative or pharmaceutically acceptable salt thereof according to claim 1, which is selected from the group consisting of:
1-cyclopentyl-4-{4-[4-(pyrrolidin-1-ylcarbonyl)-1H-pyrazol-1-yl]phenoxy}piperidine,
1-ethyl-4-{4-[4-(pyrrolidin-1-ylcarbonyl)-1H-pyrazol-1-yl]phenoxy}piperidine,
1-(1-methylethyl)-4-{4-[4-(pyrrolidin-1-ylcarbonyl)-1H-pyrazol-1-yl]phenoxy}piperidine,
1-cyclohexyl-4-{4-[4-(pyrrolidin-1-ylcarbonyl)-1H-pyrazol-1-yl]phenoxy}piperidine,
1-(2-methylcyclopentyl)-4-{4-[4-(pyrrolidin-1-ylcarbonyl)-1H-pyrazol-1-yl]phenoxy}piperidine,
1-(cyclopropylmethyl)-4-{4-[4-(pyrrolidin-1-ylcarbonyl)-1H-pyrazol-1-yl]phenoxy}piperidine,
1-(3-methyl-2-buten-1-yl)-4-{4-[4-(pyrrolidin-1-ylcarbonyl)-1H-pyrazol-1-yl]phenoxy}piperidine,
1-(cyclobutylmethyl)-4-{4-[4-(pyrrolidin-1-ylcarbonyl)-1H-pyrazol-1-yl]phenoxy}piperidine,
1-(cyclopentylmethyl)-4-{4-[4-(pyrrolidin-1-ylcarbonyl)-1H-pyrazol-1-yl]phenoxy}piperidine,
2-(4-{4-[4-(pyrrolidin-1-ylcarbonyl)-1H-pyrazol-1-yl]phenoxy}piperidin-1-yl)ethanol,
3-(4-{4-[4-(pyrrolidin-1-ylcarbonyl)-1H-pyrazol-1-yl]phenoxy}piperidin-1-yl)cyclopentanol,
ethyl 1-{4-[(1-cyclobutylpiperidin-4-yl)oxy]phenyl}-1H-pyrazole-4-carboxylic acid,
ethyl 1-{4-[(1-cyclopentylpiperidin-4-yl)oxy]phenyl}-1H-pyrazole-4-carboxylic acid,
1-cyclobutyl-4-{4-[4-(pyrrolidin-1-ylcarbonyl)-1H-pyrazol-1-yl]phenoxy}piperidine,
1-[(1-{4-[(1-cyclopentylpiperidin-4-yl)oxy]phenyl}-1H-pyrazol-4-yl)carbonyl]pyrrolidin-3-ol,
1-{4-[(1-cyclobutylpiperidin-4-yl)oxy]phenyl}-N-cyclopentyl-1H-pyrazole-4-carboxamide,
1-{4-[(1-cyclobutylpiperidin-4-yl)oxy]phenyl}-N-methyl-1H-pyrazole-4-carboxamide,
1-{4-[(1-cyclobutylpiperidin-4-yl)oxy]phenyl}-N-(2,2,2-trifluoroethyl)-1H-pyrazole-4-carboxamide,
1-{4-[(1-cyclobutylpiperidin-4-yl)oxy]phenyl}-N-(cyclohexylmethyl)-1H-pyrazole-4-carboxamide,
1-cyclobutyl-4-{4-[4-(piperidin-1-ylcarbonyl)-1H-pyrazol-1-yl]phenoxy}piperidine,
1-cyclobutyl-4-(4-{4-[(2-methylpyrrolidin-1-yl)carbonyl]-1H-pyrazol-1-yl}phenoxy)piperidine,
1-{4-[(1-cyclobutylpiperidin-4-yl)oxy]phenyl}-N,N-diethyl-1H-pyrazole-4-carboxamide,
N-tert-butyl-1-{4-[(1-cyclobutylpiperidin-4-yl)oxy]phenyl}-1H-pyrazole-4-carboxamide,
1-{4-[(1-cyclobutylpiperidin-4-yl)oxy]phenyl}-N,N-dimethyl-1H-pyrazole-4-carboxamide,
4-[(1-{4-[(1-cyclobutylpiperidin-4-yl)oxy]phenyl}-1H-pyrazol-4-yl)carbonyl]morpholine,
1-[(1-{4-[(1-cyclobutylpiperidin-4-yl)oxy]phenyl}-1H-pyrazol-4-yl)carbonyl]-4-methylpiperazine,
1-{4-[(1-cyclobutylpiperidin-4-yl)oxy]phenyl}-N-cyclohexyl-N-methyl-1H-pyrazole-4-carboxamide,
1-{4-[(1-cyclobutylpiperidin-4-yl)oxy]phenyl}-N-methyl-N-(2-methylpropyl)-1H-pyrazole-4-carboxamide,
1-cyclobutyl-4-(4-{4-[(3,3-difluoropyrrolidin-1-yl)carbonyl]-1H-pyrazol-1-yl}phenoxy)piperidine,
1-{4-[(1-cyclobutylpiperidin-4-yl)oxy]phenyl}-N-(4-fluorophenyl)-1H-pyrazole-4-carboxamide,
1-{4-[(1-cyclobutylpiperidin-4-yl)oxy]phenyl}-N-(4-fluorobenzyl)-1H-pyrazole-4-carboxamide,
4-{4-[4-(azetidin-1-ylcarbonyl)-1H-pyrazol-1-yl]phenoxy}-1-cyclopentylpiperidine,
4-[(1-{4-[(1-cyclopentylpiperidin-4-yl)oxy]phenyl}-1H-pyrazol-4-yl)carbonyl]morpholine,
1-cyclopentyl-4-(4-{4-[(3,3-difluoropyrrolidin-1-yl)carbonyl]-1H-pyrazol-1-yl}phenoxy)piperidine,
N-cyclopentyl-1-{4-[(1-cyclopentylpiperidin-4-yl)oxy]phenyl}-1H-pyrazole-4-carboxamide,
N-cyclohexyl-1-{4-[(1-cyclopentylpiperidin-4-yl)oxy]phenyl}-1H-pyrazole-4-carboxamide,
N-benzyl-1-{4-[(1-cyclopentylpiperidin-4-yl)oxy]phenyl}-1H-pyrazole-4-carboxamide,
1-{4-[(1-cyclopentylpiperidin-4-yl)oxy]phenyl}-N-(4-fluorobenzyl)-1H-pyrazole-4-carboxamide,
1-{4-[(1-cyclopentylpiperidin-4-yl)oxy]phenyl}-N-(3-fluorobenzyl)-1H-pyrazole-4-carboxamide,
1-{4-[(1-cyclopentylpiperidin-4-yl)oxy]phenyl}-N-(2-fluorobenzyl)-1H-pyrazole-4-carboxamide,
1-{4-[(1-cyclopentylpiperidin-4-yl)oxy]phenyl}-N-phenyl-1H-pyrazole-4-carboxamide,
1-{4-[(1-cyclopentylpiperidin-4-yl)oxy]phenyl}-N-(4-fluorophenyl)-1H-pyrazole-4-carboxamide,
1-{4-[(1-cyclopentylpiperidin-4-yl)oxy]phenyl}-N-(4-methylphenyl)-1H-pyrazole-4-carboxamide,
1-{4-[(1-cyclopentylpiperidin-4-yl)oxy]phenyl}-N-(4-methoxyphenyl)-1H-pyrazole-4-carboxamide,
1-{4-[(1-cyclopentylpiperidin-4-yl)oxy]phenyl}-N-[4-(pyrrolidin-1-ylcarbonyl)phenyl]-1H-pyrazole-4-carboxamide,
N-cyclopentyl-1-{4-[(1-cyclopentylpiperidin-4-yl)oxy]phenyl}-N-methyl-1H-pyrazole-4-carboxamide,
ethyl 4-{[(1-{4-[(1-cyclopentylpiperidin-4-yl)oxy]phenyl}-1H-pyrazol-4-yl)carbonyl]amino}butanoic acid,
tert-butyl 4-{[(1-{4-[(1-cyclopentylpiperidin-4-yl)oxy]phenyl]-1H-pyrazol-4-yl]carbonyl}amino]butanoic acid,
4-{[(1-{4-[(1-cyclopentylpiperidin-4-yl)oxy]phenyl}-1H-pyrazol-4-yl)carbonyl]amino}butanoic acid,
1-tert-butyl-4-{4-[4-(pyrrolidin-1-ylcarbonyl)-1H-pyrazol-1-yl]phenoxy}piperidine,
1-{4-[(1-cyclopropylpiperidin-4-yl)oxy]phenyl}-N-(4-fluorophenyl)-1H-pyrazole-4-carboxamide,
N-(4-fluorophenyl)-1-(4-{[1-(1-methylethyl)piperidin-4-yl]oxy}phenyl)-1H-pyrazole-4-carboxamide,
1-{4-[(1-cyclopentylpyrrolidin-3-yl)oxy]phenyl}-4-(pyrrolidin-1-ylcarbonyl)-1H-pyrazole,
1-{4-[(1-cyclobutylpyrrolidin-3-yl)oxy]phenyl}-4-(pyrrolidin-1-ylcarbonyl)-1H-pyrazole,
1-cyclopentyl-4-{3-fluoro-4-[4-(pyrrolidin-1-ylcarbonyl)-1H-pyrazol-1-yl]phenoxy}piperidine,
1-cyclobutyl-4-{3-fluoro-4-[4-(pyrrolidin-1-ylcarbonyl)-1H-pyrazol-1-yl]phenoxy}piperidine,
1-cyclopentyl-4-{2-fluoro-4-[4-(pyrrolidin-1-ylcarbonyl)-1H-pyrazol-1-yl]phenoxy}piperidine,
1-cyclobutyl-4-{2-fluoro-4-[4-(pyrrolidin-1-ylcarbonyl)-1H-pyrazol-1-yl]phenoxy}piperidine,
1-cyclopentyl-4-{3-methyl-4-[4-(pyrrolidin-1-ylcarbonyl)-1H-pyrazol-1-yl]phenoxy}piperidine,
1-cyclobutyl-4-{3-methyl-4-[4-(pyrrolidin-1-ylcarbonyl)-1H-pyrazol-1-yl]phenoxy}piperidine,
1-{4-[(1-cyclobutylpiperidin-4-yl)oxy]phenyl}-1H-pyrazole-4-carboxamide,
1-{4-[(1-cyclopentylpiperidin-4-yl)oxy]phenyl}-1H-pyrazole-4-carbonitrile,
1-cyclobutyl-4-[4-(4-nitro-1H-pyrazol-1-yl)phenoxy]piperidine,
4-chloro-N-(1-{4-[(1-cyclobutylpiperidin-4-yl)oxy]phenyl}-1H-pyrazol-4-yl)butaneamide,
1-(1-{4-[(1-cyclopentylpiperidin-4-yl)oxy]phenyl}-1H-pyrazol-4-yl)pyrrolidin-2-one,
2-chloroethyl {1-[4-(1-cyclobutylpiperidin-4-yloxy)phenyl]-1H-pyrazol-4-yl}-carbamic acid,
3-(1-{4-[(1-cyclopentylpiperidin-4-yl)oxy]phenyl}-1H-pyrazol-4-yl)-1,3-oxazolidin-2-one, and
{1-[4-(1-cyclobutylpiperidin-4-yloxy)phenyl]-1H-pyrazol-3-yl}pyrrolidin-1-yl-methanone.

4. A pharmaceutical preparation, which comprises the pyrazole derivative or pharmaceutically acceptable salt thereof according to any one of claims 1 to 3 as an active ingredient.

5. The pharmaceutical preparation according to claim 4, which is a histamine H3 receptor antagonist.

6. A prophylactic or therapeutic agent for dementia, Alzheimer's disease, attention-deficit hyperactivity disorder, schizophrenia, epilepsy, central convulsion, eating disorders, obesity, diabetes, hyperlipidemia, sleep disorders, narcolepsy, sleep apnea syndrome, circadian rhythm disorder, depression or allergic rhinitis, which comprises the pyrazole derivative or pharmaceutically acceptable salt thereof according to any one of claims 1 to 3 as an active ingredient.
